(19) United States
(12) Patent Application Publication
(10) Pub. No.: US 2024/0285222 A1
(43) Pub. Date: Aug. 29, 2024

(54) SYSTEMS AND METHODS FOR MODIFYING PAIN SENSITIVITY

(71) Applicant: Translation Holdings LLC, Vestavia Hills, AL (US)

(72) Inventors: Steven Rothenberg, Vestavia Hills, AL (US); Jacob Taylor, Jamaica Plain, MA (US)

(21) Appl. No.: 18/574,506

(22) PCT Filed: Jun. 28, 2022
(86) PCT No.: PCT/US22/35338

§ 371 Date: Dec. 27, 2023

Related U.S. Application Data
(60) Provisional application No. 63/202,875, filed on Jun. 28, 2021.

(51) Int. Cl. A61B 5/374; A61B 5/00

(52) U.S. Cl. CPC A61B 5/374; A61B 5/0022; A61B 5/4827; A61B 5/7275

(57) ABSTRACT

Described herein are systems and methods for modifying pain sensitivity in a subject. Example systems can include a plurality of sensors configured to detect electroencephalography (EEG) signals in the subject, and a processor communicably coupled to the plurality of sensors. The processor can be configured to receive first EEG signals from the sensors and determine, based on the first EEG signals, at least one of (i) a first value for a predicted pain sensitivity (PPS) associated with the subject or (ii) a second value for a peak alpha frequency (PAF) associated with the subject. The processor can be further configured to receive second EEG signals from the sensors and provide feedback to the subject when a characteristic of the second EEG-signals indicates a. reduced pain sensitivity.

### CROSS REFERENCE TO RELATED APPLICATIONS

This PCT application claims priority to U.S. Provisional Application No. 63/202,875, filed Jun. 28, 2021, the contents of which are incorporated herein by reference in its entirety.

### TECHNICAL FIELD

The following disclosure is directed to methods and systems for modifying pain sensitivity in a subject and, more specifically, methods and systems for modifying pain sensitivity based on electroencephalography (EEG) signals of a subject.

### BACKGROUND

Prolonged pain, including chronic pain, is conventionally managed by pharmaceuticals prescribed by physicians to patients. These pharmaceuticals can carry undesirable side effects with unknown impact on long-term patient health. In some cases, more invasive methods for mitigating chronic pain include surgeries or implants, which can be expensive and risky for patients. Examples of chronic pain include musculoskeletal pain (e.g., in a person's knees, hips, joints, etc.), neuropathic pain (e.g., diabetic neuropathy, pain associated with post-shingles, reflex sympathetic dystrophy, cancer pain, etc.), post-surgical pain, nociplastic pain (e.g. central sensitization) and pain associated with neurological disorders (e.g., anxiety, depression, attention deficit hyperactivity disorder (ADHD), etc.). For example, endometriosis is a condition affecting one in ten women of reproductive age and is characterized by chronic pelvic pain that is associated with abnormal sensitivity to pain, often unrelated to endometrial implant location. Typical surgical and hormonal treatments are found to be expensive and often ineffective.

### SUMMARY

Described herein are systems and methods for modifying pain sensitivity in a subject based on the subject's EEG signals.

Disclosed herein, in one aspect, is a system for modifying pain sensitivity in a subject, the system comprising: a) a plurality of sensors configured to detect electroencephalography (EEG) signals in the subject; and b) a processor in operative communication with the plurality of sensors and configured to: 1) receive first EEG signals from the plurality of sensors; determine, based on the first EEG signals, (i) a first predicted pain sensitivity (PPS) associated with the subject, and/or (ii) a first peak alpha frequency (PAF) associated with the subject; 2) receive second EEG signals from the sensors; and 3) provide feedback to the subject when a characteristic of the second EEG signals correlates with a second predicted pain sensitivity that is lower than the first predicted pain sensitivity.

In some embodiments, the processor is configured to determine that the pain sensitivity of the subject is modified based on at least one of the characteristic, the first PPS, and the first PAF. In some embodiments, the first PAF is from about 8 Hz to about 12 Hz. In some embodiments, the characteristic of the second EEG signals comprises a second PAF associated with the subject, and wherein the second PPS is lower than the first PPS when the second PAF is greater than the first PAF.

In some embodiments, the processor is configured to: a) receive third EEG signals from the sensors subsequent to providing the feedback; b) determine, based on the third EEG signals, (i) a third PPS, and/or (ii) a third PAF; and c) compare (i) the third PPS to the first PPS, and/or (ii) the third PAF to the first PAF, to determine whether the pain sensitivity in the subject is modified. In some embodiments, the modified pain sensitivity correlates with (i) the third PPS being lower than first PPS by a first minimum threshold, or (ii) the third PAF being greater than the first PAF by a second minimum threshold. In some embodiments, the first minimum threshold and/or the second minimum threshold is from at least about 5% to at least about 40%. In some embodiments, the second minimum threshold is from about at least about 0.01 Hz to about 1.0 Hz, such as at least about 0.02 Hz, 0.04 Hz, 0.05 Hz, 0.1 Hz, 0.25 Hz, 0.5 Hz, 0.75 Hz, or 1.0 Hz.

In some embodiments, the processor determines the first PPS, the second PPS, and/or the third PPS based on one or more of age, gender, health history, past PPS and/or PAF data, etc. In some embodiments, the first PPS, the second PPS, and/or the third PPS is based on Fourier transforms of the received corresponding first EEG signals, the second EEG signals, and/or the third EEG signals, each in an alpha frequency range of about 8 Hz to about 12 Hz. In some embodiments, the first PPS is based on the first PAF. In some embodiments, the first PPS, the second PPS, and/or the third PPS is a number on a predetermined scale from 0 to 100.

In some embodiments, the feedback to the subject comprises an auditory stimulus, a visual stimulus, a haptic stimulus, an electrical stimulus, a magnetic stimulus, an ultrasonic stimulus, or a combination thereof.

In some embodiments, the system further comprises a computing device configured to provide at least one of the auditory stimulus and the visual stimulus. In some embodiments, the computing device comprises a laptop, desktop, and/or a mobile device such as a smart phone, tablet, smartwatch, etc. In some embodiments, the computing device comprises the processor. In some embodiments, the computing device is in operative communication with the processor. In some embodiments, the auditory stimulus comprises a sound or tone having a prescribed loudness and/or pitch. In some embodiments, the auditory stimulus comprises a change in a loudness and/or pitch of a sound or tone as compared with a previously provided sound or tone. In some embodiments, the visual stimulus comprises an image depicted on a display of the computing device. In some embodiments, the visual stimulus comprises a change in a depicted image, wherein the change comprises a change in one or more of color, shape, size, and/or pattern of the depicted image. In some embodiments, the visual stimulus comprises a change from a less pleasing image to a more pleasing image. In some embodiments, the system further comprises an apparatus configured to apply at least one of a magnetic stimulus and/or an ultrasonic stimulus to a brain or a peripheral nervous system of the subject.

In some embodiments, the feedback correlates only with a decreasing PPS.

In some embodiments, the feedback includes points based on the decreasing PPS that is optionally cumulative with each successive training session. In some embodiments, the plurality of sensors comprise electrodes configured to be positioned on a head of the subject. In some embodiments, the plurality of sensors are provided with a headwear assembly, such as a headband, hat, or helmet.

In some embodiments, the processor is part of a computing device. In some embodiments, the computing device comprises a mobile device, a desktop, a laptop, and/or a remote computing system. In some embodiments, the mobile device comprises a smartphone, a smart watch, a tablet, or any combination thereof. In some embodiments, the computing device comprises and/or is operatively coupled to a storage module coupled to the processor and configured to store the first EEG signals, the second EEG signals, the third EEG signals, the first PPS, the second PPS, the third PPS, the first PAF, the second PAF, the third PAF, or a combination thereof. In some embodiments, the computing device comprises a user interface configured to provide the feedback and/or receive input from the subject.

In some embodiments, the system further comprising: a) a first communication module coupled to the plurality of sensors and configured to transmit the first, second, and/or third EEG signals; and b) a second communication module configured to receive the first, second, and/or third EEG signals from the first communication module. In some embodiments, the second communication module is part of the computing device.

In some embodiments, the feedback has a type, and wherein the processor is further configured to provide additional feedback of any type to the subject after determining that the pain sensitivity of the subject is modified. In some embodiments, the system is configured to modify pain sensitivity (i) associated with endometriosis in the subject, (ii) as an adjuvant therapy to endometriosis related central sensitization, (iii) associated with musculoskeletal pain in the subject, (iv) associated with diabetic neuropathy in the subject, (iv) associated with shingles in the subject, (v) associated with reflex sympathetic dystrophy syndrome in the subject, (vi) associated with cancer in the subject, (vii) associated with post-surgical pain in the subject, (viii) associated with a neurological disorder in the subject, (ix) associated with anxiety in the subject, (x) associated with depression in the subject, (xi) associated with attention deficit hyperactivity disorder (ADHD) in the subject, (xii) associated with post traumatic stress disorder (PTSD), (xiii) associated with nociplastic pain, (xiv) associated with chronic pelvis pain, (xv) associated with fibromyalgia, (xvi) associated with post traumatic pain, (xvii) associated with post surgical pain, or (xvii) any combination thereof. In some embodiments, the system is configured to prevent or reduce a chronification of pain in the subject experiencing acute pain.

In some embodiments, the first EEG signals correspond to a lowest PPS score from a previous therapy session.

Disclosed herein, in another aspect, is a method for modifying pain sensitivity in a subject, the method comprising: a) receiving first EEG signals from a plurality of sensors coupled to the subject; b) determining, based on the first EEG signals, (i) a first predicted pain sensitivity (PPS) associated with the subject, and/or (ii) a first peak alpha frequency (PAF) associated with the subject; c) receiving second EEG signals from the plurality sensors; and d) providing feedback to the subject when a characteristic of the second EEG signals correlates with a second predicted pain sensitivity that is lower than the first predicted pain sensitivity.

In some embodiments, the first PAF is from about 8 Hz to about 12 Hz. In some embodiments, the characteristic of the second EEG signals comprises a second PAF associated with the subject, and wherein the second PPS is lower than the first PPS when the second PAF is greater than the first PAF.

In some embodiments, the method further comprises: a) receiving third EEG signals from the sensors subsequent to providing the feedback; b) determining, based on the third EEG signals, (i) a third PPS, and/or (ii) a third PAF; and c) comparing (i) the third PPS to the first PPS, and/or (ii) the third PAF to the first PAF, to determine whether the pain sensitivity in the subject is modified. In some embodiments, the modified pain sensitivity correlates with (i) the third PPS being lower than first PPS by a first minimum threshold, or (ii) the third PAF being greater than the first PAF by a second minimum threshold. In some embodiments, the first minimum threshold and/or the second minimum threshold is from at least about 5% to at least about 40%. In some embodiments, the second minimum threshold is from about at least about 0.01 Hz to about 1.0 Hz, such as at least about 0.02 Hz, 0.04 Hz, 0.05 Hz, 0.1 Hz, 0.25 Hz, 0.5 Hz, 0.75 Hz, or 1.0 Hz.

In some embodiments, the first PPS, the second PPS, and/or the third PPS is based on one or more of age, gender, health history, past PPS and/or PAF data, etc. In some embodiments, the first PPS, the second PPS, and/or the third PPS is based on Fourier transforms of the received corresponding first EEG signals, the second EEG signals, and/or the third EEG signals, each in an alpha frequency range of about 8 Hz to about 12 Hz. In some embodiments, the first PPS is based on the first PAF. In some embodiments, the first PPS, the second PPS, and/or the third PPS is a number on a predetermined scale from 0 to 100.

In some embodiments, the feedback to the subject comprises an auditory stimulus, a visual stimulus, a haptic stimulus, an electrical stimulus, a magnetic stimulus, an ultrasonic stimulus, or a combination thereof. In some embodiments, a computing device is configured to provide at least one of the auditory stimulus and the visual stimulus. In some embodiments, the computing device comprises a laptop, desktop, and/or a mobile device such as a smart phone, tablet, smartwatch, etc. In some embodiments, the auditory stimulus comprises a sound or tone having a prescribed loudness and/or pitch. In some embodiments, the auditory stimulus comprises a change in a loudness and/or pitch of a sound or tone as compared with a previously provided sound or tone. In some embodiments, the visual stimulus comprises an image depicted on a display of the computing device. In some embodiments, the visual stimulus comprises a change in a depicted image, wherein the change comprises a change in one or more of color, shape, size, and/or pattern of the depicted image. In some embodiments, the visual stimulus comprises a change from a less pleasing image to a more pleasing image. In some embodiments, the method further comprises using an apparatus configured to apply at least one of a magnetic stimulus and/or an ultrasonic stimulus to a brain or a peripheral nervous system of the subject.

In some embodiments, the feedback correlates only with a decreasing PPS. In some embodiments, the feedback includes points based on the decreasing PPS that is optionally cumulative with each successive training session.

In some embodiments, the feedback has a type, and wherein the processor is further configured to provide additional feedback of any type to the subject after determining that the pain sensitivity of the subject is modified. In some embodiments, the method enables pain sensitivity modification (i) associated with endometriosis in the subject, (ii) as an adjuvant therapy to endometriosis related central sensitization, (iii) associated with musculoskeletal pain in the subject, (iv) associated with diabetic neuropathy in the subject, (iv) associated with shingles in the subject, (v) associated with reflex sympathetic dystrophy syndrome in the subject, (vi) associated with cancer in the subject, (vii) associated with post-surgical pain in the subject, (viii) associated with a neurological disorder in the subject, (ix) associated with anxiety in the subject, (x) associated with depression in the subject, (xi) associated with attention deficit hyperactivity disorder (ADHD) in the subject, (xii) associated with post traumatic stress disorder (PTSD), (xiii) associated with nociplastic pain, (xiv) associated with chronic pelvis pain, (xv) associated with fibromyalgia, (xvi) associated with post traumatic pain, (xvii) associated with post surgical pain, or (xvii) any combination thereof. In some embodiments, the method prevents or reduces chronification of pain in the subject experiencing acute pain. In some embodiments, the first EEG signals correspond to a lowest PPS score from a previous therapy session.

Disclosed herein, in another embodiment, is a non-transitory computer readable medium for modifying pain sensitivity in a subject, the non-transitory computer readable medium comprising instructions that, when executed by a processor, cause the processor to perform operations including: a) receiving first EEG signals from a plurality of sensors coupled to the subject; b) determining, based on the first EEG signals, (i) a first predicted pain sensitivity (PPS) associated with the subject, and/or (ii) a first peak alpha frequency (PAF) associated with the subject; c) receiving second EEG signals from the plurality sensors; and d) providing feedback to the subject when a characteristic of the second EEG signals correlates with a second predicted pain sensitivity that is lower than the first predicted pain sensitivity.

In some embodiments, the processor is configured to determine that the pain sensitivity of the subject is modified based on at least one of the characteristic, the first PPS, and the first PAF. In some embodiments, the first PAF is from about 8 Hz to about 12 Hz. In some embodiments, the characteristic of the second EEG signals comprises a second PAF associated with the subject, and wherein the second PPS is lower than the first PPS when the second PAF is greater than the first PAF.

In some embodiments, the processor is configured to: a) receive third EEG signals from the sensors subsequent to providing the feedback; b) determine, based on the third EEG signals, (i) a third PPS, and/or (ii) a third PAF; and c) compare (i) the third PPS to the first PPS, and/or (ii) the third PAF to the first PAF, to determine whether the pain sensitivity in the subject is modified. In some embodiments, the modified pain sensitivity correlates with (i) the third PPS being lower than first PPS by a first minimum threshold, or (ii) the third PAF being greater than the first PAF by a second minimum threshold. In some embodiments, the first minimum threshold and/or the second minimum threshold is from at least about 5% to at least about 40%. In some embodiments, the second minimum threshold is from about at least about 0.01 Hz to about 1.0 Hz, such as at least about 0.02 Hz, 0.04 Hz, 0.05 Hz, 0.1 Hz, 0.25 Hz, 0.5 Hz, 0.75 Hz, or 1.0 Hz.

In some embodiments, the processor determines the first PPS, the second PPS, and/or the third PPS based on one or more of age, gender, health history, past PPS and/or PAF data, etc. In some embodiments, the first PPS, the second PPS, and/or the third PPS is based on Fourier transforms of the received corresponding first EEG signals, the second EEG signals, and/or the third EEG signals, each in an alpha frequency range of about 8 Hz to about 12 Hz. In some embodiments, the first PPS is based on the first PAF. In some embodiments, the first PPS, the second PPS, and/or the third PPS is a number on a predetermined scale from 0 to 100.

In some embodiments, the feedback to the subject comprises an auditory stimulus, a visual stimulus, a haptic stimulus, an electrical stimulus, a magnetic stimulus, an ultrasonic stimulus, or a combination thereof.

In some embodiments, the non-transitory computer readable medium further comprises a computing device configured to provide at least one of the auditory stimulus and the visual stimulus. In some embodiments, the computing device comprises a laptop, desktop, and/or a mobile device such as a smart phone, tablet, smartwatch, etc. In some embodiments, the computing device comprises the processor. In some embodiments, the computing device is in operative communication with the processor. In some embodiments, the auditory stimulus comprises a sound or tone having a prescribed loudness and/or pitch. In some embodiments, the auditory stimulus comprises a change in a loudness and/or pitch of a sound or tone as compared with a previously provided sound or tone. In some embodiments, the visual stimulus comprises an image depicted on a display of the computing device. In some embodiments, the visual stimulus comprises a change in a depicted image, wherein the change comprises a change in one or more of color, shape, size, and/or pattern of the depicted image. In some embodiments, the visual stimulus comprises a change from a less pleasing image to a more pleasing image. In some embodiments, the non-transitory computer readable medium further comprises an apparatus configured to apply at least one of a magnetic stimulus and/or an ultrasonic stimulus to a brain or a peripheral nervous system of the subject.

In some embodiments, the feedback correlates only with a decreasing PPS.

In some embodiments, wherein the feedback includes points based on the decreasing PPS that is optionally cumulative with each successive training session. In some embodiments, the plurality of sensors comprise electrodes configured to be positioned on a head of the subject. In some embodiments, the plurality of sensors are provided with a headwear assembly, such as a headband, hat, or helmet.

In some embodiments, the processor is part of a computing device. In some embodiments, the computing device comprises a mobile device, a desktop, a laptop, and/or a remote computing system. In some embodiments, the mobile device comprises a smartphone, a smart watch, a tablet, or any combination thereof. In some embodiments, the computing device comprises and/or is operatively coupled to a storage module coupled to the processor and configured to store the first EEG signals, the second EEG signals, the third EEG signals, the first PPS, the second PPS, the third PPS, the first PAF, the second PAF, the third PAF, or a combination thereof. In some embodiments, the computing device comprises a user interface configured to provide the feedback and/or receive input from the subject.

In some embodiments, the non-transitory computer readable medium further comprising: a) a first communication module coupled to the plurality of sensors and configured to transmit the first, second, and/or third EEG signals; and b) a second communication module configured to receive the first, second, and/or third EEG signals from the first communication module. In some embodiments, the second communication module is part of the computing device.

In some embodiments, the feedback has a type, and wherein the processor is further configured to provide additional feedback of any type to the subject after determining that the pain sensitivity of the subject is modified. In some embodiments, the non-transitory computer readable medium is configured to modify pain sensitivity (i) associated with endometriosis in the subject, (ii) as an adjuvant therapy to endometriosis related central sensitization, (iii) associated with musculoskeletal pain in the subject, (iv) associated with diabetic neuropathy in the subject, (iv) associated with shingles in the subject, (v) associated with reflex sympathetic dystrophy syndrome in the subject, (vi) associated with cancer in the subject, (vii) associated with post-surgical pain in the subject, (viii) associated with a neurological disorder in the subject, (ix) associated with anxiety in the subject, (x) associated with depression in the subject, (xi) associated with attention deficit hyperactivity disorder (ADHD) in the subject, (xii) associated with post traumatic stress disorder (PTSD), (xiii) associated with nociplastic pain, (xiv) associated with chronic pelvis pain, (xv) associated with fibromyalgia, (xvi) associated with post traumatic pain, (xvii) associated with post surgical pain, or (xvii) any combination thereof. In some embodiments, the non-transitory computer readable medium is configured to prevent or reduce a chronification of pain in the subject experiencing acute pain.

In some embodiments, the first EEG signals correspond to a lowest PPS score from a previous therapy session.

Disclosed herein, in another aspect, is a non-transitory computer readable medium for modifying pain sensitivity in a subject, the non-transitory computer readable medium comprising instructions that, when executed by a processor, cause the processor to perform operations including: a) receiving first EEG signals from a plurality of sensors coupled to the subject; b) determining, based on the first EEG signals, (i) a first predicted pain sensitivity (PPS) associated with the subject, and/or (ii) a first peak alpha frequency (PAF) associated with the subject; c) correlating an entrainment regimen based on the first PPS and/or the first PAF; and d) providing the entrainment regiment to the subject.

In some embodiments, the operations further include: a) receiving second EEG signals from the plurality of sensors or a different plurality of sensors coupled to the subject; b) determining, based on the second EEG signals, (i) a second PPS associated with the subject, and/or (ii) a second PAF associated with the subject; and c) identifying an effectiveness of the entrainment regimen.

In some embodiments, the operations further includes modifying the entrainment regimen based on the identified effectiveness. In some embodiments, the identified effectiveness is based on i) the second PPS being lower than the first PPS by a first minimum threshold, and/or ii) the second PAF being greater than the first PAF by a second minimum threshold. In some embodiments, the first minimum threshold and/or the second minimum threshold is from at least about 5% to at least about 40%. In some embodiments, the second minimum threshold is from about at least about 0.01 Hz to about 1.0 Hz, such as at least about 0.02 Hz, 0.04 Hz, 0.05 Hz, 0.1 Hz, 0.25 Hz, 0.5 Hz, 0.75 Hz, or 1.0 Hz.

In some embodiments, the entrainment regimen comprises one or more audio stimuli and/or one or more video stimuli. In some embodiments, the one or more audio stimuli comprises a volume of a tone, musical track, and/or other sound at a prescribed frequency based on the first PAF and/or the first PPS. In some embodiments, the one or more audio stimuli comprises sub perceptible background tone at the prescribed frequency. In some embodiments, the one or more audio stimuli comprises a beat frequency wherein two tones have a difference in frequency of the prescribed frequency. In some embodiments, the one or more video stimuli comprises a flicker and/or oscillation provided on a display device, wherein the flicker and/or oscillation is provided at the prescribed frequency. In some embodiments, the one or more video stimuli comprises a flicker and/or oscillation provided on a display device, wherein the flicker and/or oscillation is provided at the prescribed frequency.

In some embodiments, the entrainment regimen comprises providing a vibrotactile stimulation at the prescribed frequency. In some embodiments, the prescribed frequency is based on a minimum PAF that correlates with a PPS greater than the first PPS. In some embodiments, the prescribed frequency is from about 10 Hz to about 12 Hz.

In some embodiments, the processor is part of a computing device. In some embodiments, the computing device comprises a mobile device, a desktop, a laptop, and/or a remote computing system. In some embodiments, the mobile device comprises a smartphone, a smart watch, a tablet, or any combination thereof. In some embodiments, the computing device comprises and/or is operatively coupled to a storage module coupled to the processor and configured to store the first EEG signals, the second EEG signals, the first PPS, the second PPS, the first PAF, the second PAF, or a combination thereof. In some embodiments, the computing device comprises a user interface configured to provide the feedback and/or receive input from the subject. In some embodiments, the entrainment regimen is provided by the computing device.

In some embodiments, the processor determines the first PPS and/or the second PPS based on one or more of age, gender, health history, past PPS and/or PAF data, etc. In some embodiments, wherein the first PPS and/or the second PPS is based on Fourier transforms of the received corresponding first EEG signals and/or the second EEG signals, each in an alpha frequency range of about 8 Hz to about 12 Hz.

In some embodiments, the first PPS is based on the first PAF. In some embodiments, the first PPS and/or the second PPS is a number on a predetermined scale from 0 to 100. In some embodiments, the plurality of sensors comprise electrodes configured to be positioned on a head of the subject. In some embodiments, the plurality of sensors are provided with a headwear assembly, such as a headband, hat, or helmet.

In some embodiments, the computing device comprises and/or is operatively coupled to a storage module coupled to the processor and configured to store the first EEG signals, the second EEG signals, the third EEG signals, the first PPS, the second PPS, the third PPS, the first PAF, the second PAF, the third PAF, or a combination thereof. In some embodiments, the computing device comprises a user interface configured to receive input from the subject.

In some embodiments, the non-transitory computer readable medium further comprising: a) a first communication module coupled to the plurality of sensors and configured to transmit the first and/or second EEG signals, and b) a second communication module configured to receive the first and/or second EEG signals from the first communication module. In some embodiments, the second communication module is part of the computing device.

In some embodiments the non-transitory computer readable medium is configured to modify pain sensitivity (i) associated with endometriosis in the subject, (ii) as an adjuvant therapy to endometriosis related central sensitization, (iii) associated with musculoskeletal pain in the subject, (iv) associated with diabetic neuropathy in the subject, (iv) associated with shingles in the subject, (v) associated with reflex sympathetic dystrophy syndrome in the subject, (vi) associated with cancer in the subject, (vii) associated with post-surgical pain in the subject, (viii) associated with a neurological disorder in the subject, (ix) associated with anxiety in the subject, (x) associated with depression in the subject, (xi) associated with attention deficit hyperactivity disorder (ADHD) in the subject, (xii) associated with post traumatic stress disorder (PTSD), (xiii) associated with nociplastic pain, (xiv) associated with chronic pelvis pain, (xv) associated with fibromyalgia, (xvi) associated with post traumatic pain, (xvii) associated with post surgical pain, or (xvii) any combination thereof. In some embodiments, the non-transitory computer readable medium is configured to prevent or reduce a chronification of pain in the subject experiencing acute pain. In some embodiments, the first EEG signals correspond to a lowest PPS score from a previous therapy session.

Disclosed herein, in another aspect, is a method for modifying pain sensitivity in a subject, the method comprising: a) receiving first EEG signals from a plurality of sensors coupled to the subject; b) determining, based on the first EEG signals, (i) a first predicted pain sensitivity (PPS) associated with the subject, and/or (ii) a first peak alpha frequency (PAF) associated with the subject; and c) correlating an entrainment regimen based on the first PPS and/or the first PAF; and d) providing the entrainment regiment to the subject.

In some embodiments, the method further comprises a) receiving second EEG signals from the plurality of sensors or a different plurality of sensors coupled to the subject; b) determining, based on the second EEG signals, (i) a second PPS associated with the subject, and/or (ii) a second PAF associated with the subject; and c) identifying an effectiveness of the entrainment regimen.

In some embodiments, the method further comprises modifying the entrainment regimen based on the identified effectiveness. In some embodiments, the identified effectiveness is based on i) the second PPS being lower than the first PPS by a first minimum threshold, and/or ii) the second PAF being greater than the first PAF by a second minimum threshold. In some embodiments, the first minimum threshold and/or the second minimum threshold is from at least about 5% to at least about 40%. In some embodiments, the second minimum threshold is from about at least about 0.01 Hz to about 1.0 Hz, such as at least about 0.02 Hz, 0.04 Hz, 0.05 Hz, 0.1 Hz, 0.25 Hz, 0.5 Hz, 0.75 Hz, or 1.0 Hz.

In some embodiments, the entrainment regimen comprises one or more audio stimuli and/or one or more video stimuli. In some embodiments, the one or more audio stimuli comprises a volume of a tone, musical track, and/or other sound at a prescribed frequency based on the first PAF and/or the first PPS. In some embodiments, the one or more audio stimuli comprises sub perceptible background tone at the prescribed frequency. In some embodiments, the one or more audio stimuli comprises a beat frequency wherein two tones have a difference in frequency of the prescribed frequency. In some embodiments, the one or more video stimuli comprises a flicker and/or oscillation provided on a display device, wherein the flicker and/or oscillation is provided at the prescribed frequency. In some embodiments, the one or more video stimuli comprises a flicker and/or oscillation provided on a display device, wherein the flicker and/or oscillation is provided at the prescribed frequency.

In some embodiments, the entrainment regimen comprises providing a vibrotactile stimulation at the prescribed frequency. In some embodiments, the prescribed frequency is based on a minimum PAF that correlates with a PPS greater than the first PPS. In some embodiments, the prescribed frequency is from about 10 Hz to about 12 Hz. In some embodiments, the first PPS and/or the second PPS is based on one or more of age, gender, health history, past PPS and/or PAF data, etc. In some embodiments, the first PPS and/or the second PPS is based on Fourier transforms of the received corresponding first EEG signals and/or the second EEG signals, each in an alpha frequency range of about 8 Hz to about 12 Hz. In some embodiments, the first PPS is based on the first PAF. In some embodiments, the first PPS and/or the second PPS is a number on a predetermined scale from 0 to 100.

In some embodiments, the method enables pain sensitivity modification (i) associated with endometriosis in the subject, (ii) as an adjuvant therapy to endometriosis related central sensitization, (iii) associated with musculoskeletal pain in the subject, (iv) associated with diabetic neuropathy in the subject, (iv) associated with shingles in the subject, (v) associated with reflex sympathetic dystrophy syndrome in the subject, (vi) associated with cancer in the subject, (vii) associated with post-surgical pain in the subject, (viii) associated with a neurological disorder in the subject, (ix) associated with anxiety in the subject, (x) associated with depression in the subject, (xi) associated with attention deficit hyperactivity disorder (ADHD) in the subject, (xii) associated with post traumatic stress disorder (PTSD), (xiii) associated with nociplastic pain, (xiv) associated with chronic pelvis pain, (xv) associated with fibromyalgia, (xvi) associated with post traumatic pain, (xvii) associated with post surgical pain, or (xvii) any combination thereof. In some embodiments, the method is configured to prevent or reduce a chronification of pain in the subject experiencing acute pain.

In some embodiments, the first EEG signals correspond to a lowest PPS score from a previous therapy session.

In some embodiments, the entrainment regiment is provided using a computing device. In some embodiments, the computing device comprises a mobile device, a desktop, a laptop, and/or a remote computing system. In some embodiments, the mobile device comprises a smartphone, a smart watch, a tablet, or any combination thereof.

### BRIEF DESCRIPTION OF THE DRAWINGS

FIG. 1 is a plot illustrating EEG signal power as a function of frequency.

FIG. 2A is a diagram of an example data flow for modifying pain sensitivity in a patient.

FIG. 2B is a diagram illustrating an example shift in predicted pain sensitivity.

FIG. 2C is a plot of example data of a shift in pain sensitivity before, during, and after training.

FIG. 3 is a diagram of an example system for modifying pain sensitivity in a subject.

FIG. 4 is a diagram of an example method for modifying pain sensitivity in a subject.

FIG. 5 is a block diagram of an example computer system that may be used in implementing the systems and methods described herein.

FIG. 6 is a diagram of an example of another method for modifying pain sensitivity in a subject.

FIG. 7 depicts results from a protocol for 3 participants comparing an effect on modifying pain sensitivity when using neurofeedback or a sham method.

FIG. 8 depicts power results for Participant #2 from the protocol in FIG. 7.

FIG. 9 depicts a pain rating for Participant #2 from the protocol in FIG. 7.

### DETAILED DESCRIPTION

Disclosed herein are exemplary embodiments of systems and methods for modifying pain sensitivity in a human subject. In particular, the systems and methods may use a plurality of sensors (e.g., superficial electrodes) to measure brain waves to modify pain sensitivity for treating acute or chronic pain and/or prevent chronic pain from developing in a patient.

In an example use case, a subject may be presented with a headband that includes electrodes for collecting EEG signals. The headband may be connected to a processor (e.g., as part of a handheld device, as part of a computing device, etc.) and may be used for therapy for modifying a subject's pain sensitivity. Information about the therapy (e.g., including instructions, feedback signals, visual cues, etc.) may be provided to the user interface of a mobile device such that the subject may access the information.

The example systems and methods described herein can provide a non-invasive, long-term solution for chronic pain, which affects millions of people nationwide. As described further below, patients can be trained (e.g., as part of a therapy) to reduce their pain sensitivity by receiving neurofeedback based on their EEG signals.

### Predicted Pain Sensitivity

Pain sensitivity in a subject may be predicted by collecting data of certain oscillations in their resting EEG signals and analyzing the frequency of the oscillations in the EEG signals. The result may be referred to as the “predicted pain sensitivity” (PPS) of a subject. Examples of predicting pain sensitivity may be found in International Application Publication No. WO2019/090041 A1 published on May 9, 2019 and titled “Method for Predicting Pain Sensitivity.”

In some embodiments, PPS is determined based a database (e.g., a normative database) of pain sensitivity data. In some embodiments, the PPS is determined based on resting EEG measurements. In some embodiments, the PPS does not need a pain stimulus for determination. The example database may include EEG signals, age information, gender, health history, family history, therapy history, demographic information, medications, pain sensitivity data, and/or PAF associated with subjects. For instance, the EEG signals in the database may include EEG signals before and/or after a medical intervention (e.g., a medical treatment, surgery, medication, psychotherapy, etc.). The database may further include the outcomes of such medical intervention (e.g., improvement in well-being, physical function, etc.). For example, EEG signals from a given patient (or groups of patients) before and after surgical operation may be collected into the database. Further, data indicating the pain medication (e.g., opioids) consumption and/or pain ratings of these patient(s) may also be collected. Note that the example database may draw on data from public sources or specifically collected data for a group of patients. In some embodiments, longitudinal resting state EEG data is used as a feature (in addition to other features in the normative database) to train a machine learning model (e.g., logistic regression, support vector machines, deep learning, etc.) that can generate the PPS. The EEG data may be from multiple points in time to refine the predicted pain sensitivity (e.g., before and/or after a specific medical procedure). In some embodiments, PPS may be derived using a Fourier transform of one or more EEG signals of the subject. The Fourier transform of the EEG signal(s) may be in an alpha frequency range of approximately 8-12 Hz (e.g., +/−1 Hz).

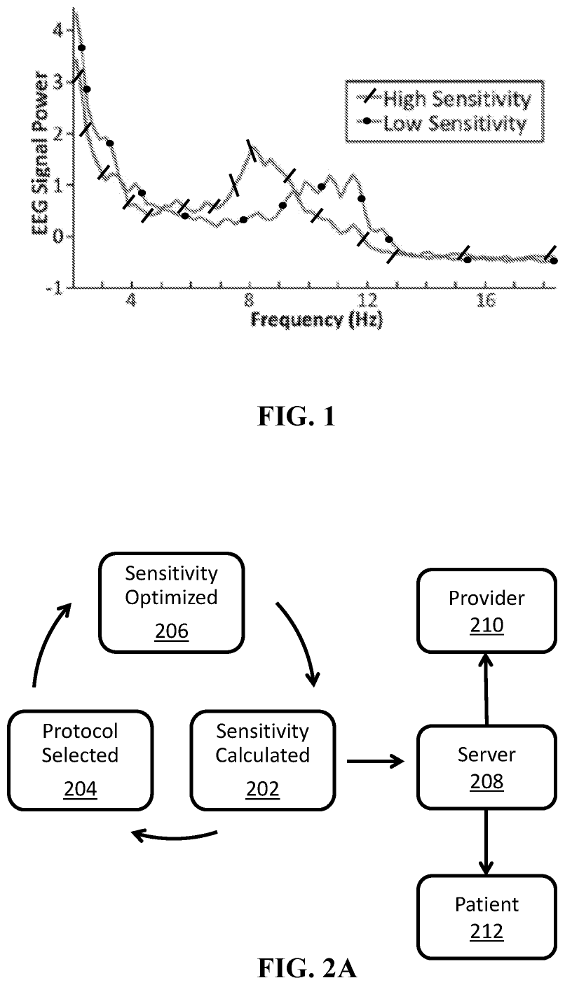

In some embodiments, PPS is determined based on a peak alpha frequency (PAF) of a subject. In some embodiments, PPS can be calculated by determining power calculations in 0.1 Hz bins and evaluating the ratio of slow alpha (summed power in the 8-9 Hz range) to fast alpha (summed power in the 10-11 Hz range). In another embodiment, PPS is calculated by assigning a correlation coefficient to sum 0.1 Hz bins across the 8-12 Hz range where positive coefficients are assigned to the slow alpha range (8-9 Hz) and negative coefficients are assigned to the fast alpha range (10-11 Hz). In some embodiments, these correlation coefficients are determined by an age and gender matched normative database. FIG. 1 illustrates EEG signal power as a function of frequency for PAF biomarker calculations. For instance, a subject having a PAF of less than 9 Hz can be classified as a subject having high sensitivity to pain (plot line 102) while a PAF of greater than 9 Hz indicates low sensitivity (plot line 104). In past studies, the PAF biomarker can differentiate between high and low pain sensitivity individuals for capsaicin heat pain (21 participants, p=0.026) (Furman et al., “Cerebral Peak Alpha Frequency Predicts Individual Differences in Pain Sensitivity,” Neuro Image, volume 167, 203-210, doi.org/10.1016/j.neuroimage.2017.11.042, Nov. 21, 2017). A study with a clinically-relevant, human model of prolonged pain (persisting for weeks) using intramuscular nerve growth factor injections demonstrated that the speed of pain-free, sensorimotor peak alpha frequency recorded during resting-state EEG predicts pain sensitivity (31 participants, p<0.01) (Furman et al., “Cerebral peak alpha frequency reflects average pain severity in a human model of sustained, musculoskeletal pain.” Neurophysiology, 122(4):1784-93, pubmed.ncbi.nlm.nih.gov/31389754/, Oct. 1, 2019). It was further found that PAF predicts an individual's pain sensitivity to multiple pain paradigms and is reliable at multiple time points. The experiments were repeated collecting the same measurement weeks apart (participants 61, p<0.01) (Furman et al., “Sensorimotor Peak Alpha Frequency Is a Reliable Biomarker of Prolonged Pain Sensitivity,” Cerebral Cortex, 30(12):6069-82, pubmed.ncbi.nlm.nih.gov/32591813/, Nov. 3, 2020).

In various embodiments, PPS is presented on a scale for use by a subject and/or heath care provider. For example, predicted pain sensitivity can be provided on a numeric scale (e.g., 0 to 10) and/or alphabetical scale (e.g., A to E, A to J, etc.). In some embodiments, the PPS is provided as a scale of 1-100 wherein 1 represents low sensitivity, and 100 represents high sensitivity.

In some embodiments, a health care provider may collect PPS data from a patient at the point of care. For example, a physician may collect PPS data of a patient after a traumatic injury (e.g., in an emergency care setting). In another example, a specialist may collect PPS data of a patient as part of disease management. In another example, a primary care provider may collect PPS data of a patient as part of routine health care. In another example, a physician may send a device configured to collect PPS data to a patient (e.g., as part of a tele-health care or prescribed therapy). In a particular example, PPS data may be collected for patients experiencing chronic pelvic pain with suspected or confirmed endometriosis. Endometriosis symptoms can be caused by pain sensitization that is often unrelated to disease burden. Sensitivity to this condition, which affects nearly 7.5 million women in the United States, may be improved by the example systems and methods described herein. As described further below, PPS and/or PAF data can be used in improving a patient's pain sensitivity.

In some embodiments, systems and methods described herein are configured to modify pain sensitivity (i) associated with endometriosis in the subject, (ii) as an adjuvant therapy to endometriosis related central sensitization, (iii) associated with musculoskeletal pain in the subject, (iv) associated with diabetic neuropathy in the subject, (iv) associated with shingles in the subject, (v) associated with reflex sympathetic dystrophy syndrome in the subject, (vi) associated with cancer in the subject, (vii) associated with post-surgical pain in the subject, (viii) associated with a neurological disorder in the subject, (ix) associated with anxiety in the subject, (x) associated with depression in the subject, (xi) associated with attention deficit hyperactivity disorder (ADHD) in the subject, (xii) associated with post traumatic stress disorder (PTSD), (xiii) associated with nociplastic pain, (xiv) associated with chronic pelvis pain, (xv) associated with fibromyalgia, (xvi) associated with post traumatic pain, (xvii) associated with post surgical pain, or (xvii) any combination thereof.

### Modifying Pain Sensitivity

### Neurofeedback

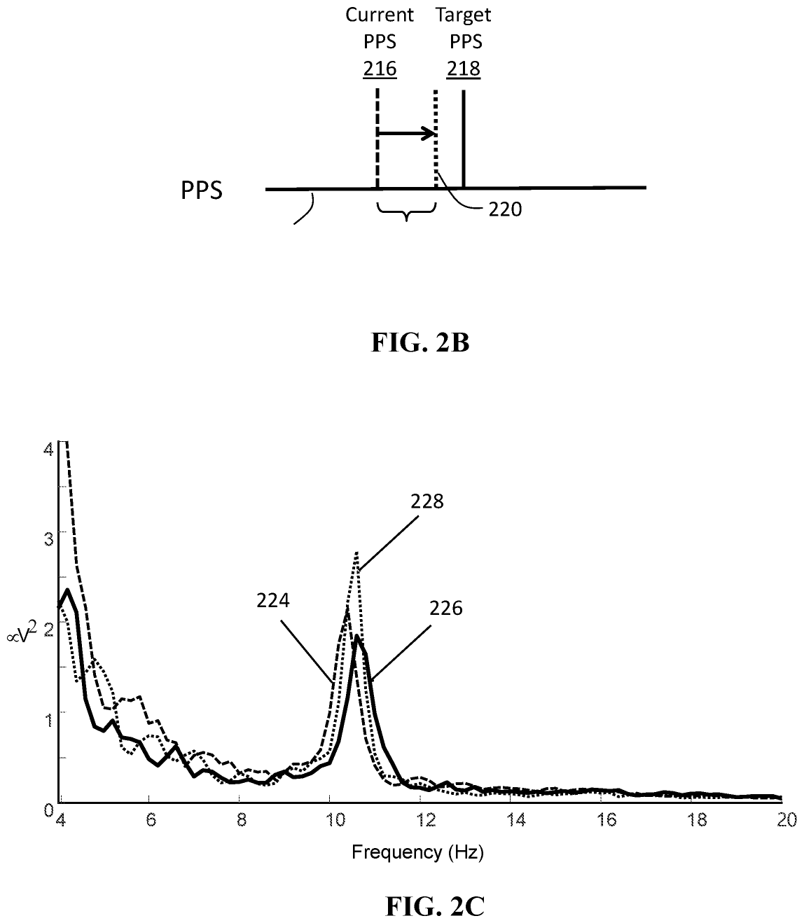

In some embodiments, a provider may prescribe neurofeedback therapy to modify a patient's pain sensitivity. For example, such therapy may be especially helpful to patients who are highly sensitive to pain (e.g., refer to example curve 102 in FIG. 1). FIG. 2A shows a high-level data flowchart for neuromodulation based on PPS and/or PAF. In process 202, the pain sensitivity of a patient is determined based on EEG signals received from sensors on the subject. Initial pain sensitivity data may be referred to as “baseline” pain sensitivity for that subject. As described further below, additional pain sensitivity data may be collected to modify the subject's pain sensitivity. In some embodiments, the neurofeedback therapy may be provided in discrete sessions, wherein each session comprises providing the neurofeedback for a duration of time and/or until a target PPS is achieved (as described herein). In some embodiments, each session may be separated by a time period. For example, some therapy sessions may be provided daily, multiple times during a day (e.g., morning, afternoon), every other day, weekly, bi-weekly, monthly, or any non-periodic schedule. As used herein, a “training” may refer to “therapy”.

In some embodiments, the baseline PPS correlates to a first recorded EEG signals during a therapy session. In some embodiments, the baseline PPS correlates to a lowest PPS in a first plurality of EEG signals recorded during a therapy session. In some embodiments, the baseline PPS is based on a previously recorded EEG signals from a previous session of therapy. For example, a previous session of therapy may be a previous date when the therapy occurred (as compared to the current therapy), or a different time period (e.g., morning vs. afternoon). In some embodiments, the previously recorded EEG signals correlate to a lowest recorded PPS from the previous session.

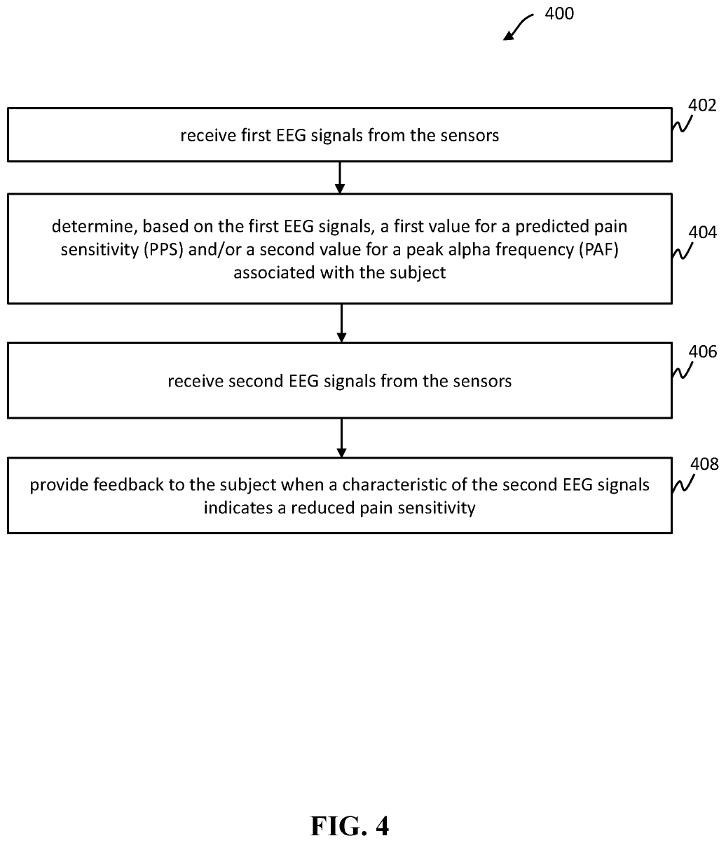

In process 204, the neuromodulation feedback (NFB) protocol is selected (e.g., as part of training or therapy). The NFB protocol may include determining amount, type, frequency, etc. of feedback based on the sensitivity data. As used herein, “training” may refer to a given session in which a subject is receiving feedback based on the subject's EEG signals (e.g., neurofeedback). In some cases, training may be part of therapy. Feedback signals (also referred to as “rewards”) are provided to the patient when received EEG signals indicate a brainwave state that has improved (e.g., above the patient's baseline PPS). Note that, in some cases, such feedback signals reinforce shifts (e.g., natural shifts, random shifts, intentional shifts, etc.) in PAF to “lock” the patient in a desired brainwave state. In some embodiments, feedback is provided when the received EEG signal reaches a target PPS and/or exceeds a reward threshold. Feedback may be provided when the received EEG signal causes the change desired in the PPS. The reward rate is the amount of feedback to provide when the patient improves their PPS and/or PAF. The reward rate may be varied. For instance, feedback may be provided to a subject upon the subject exceeding one or more predetermined thresholds and/or meeting certain targets in her/his PPS and/or PAF. In some embodiments, the reward rate can be determined (e.g., calculated, measured, etc.) to reward the subject such that the subject is motivated to stay in an improved state, e.g., a state with reduced pain sensitivity and/or increased PAF. In some embodiments, no reward is given for a period of time (e.g., from about 1 to about 30 seconds, such as about 1 second to about 15 seconds, or about 2 seconds to about 5 seconds) when the determined PPS is not higher than the baseline PPS (as described herein). In some embodiments, PAF is calculated using the first EEG signals (e.g., at approximately 8-12 Hz) from the sensors (refer to step 402 of FIG. 4). Feedback may be provided to the patient by increasing the volume of a tone when alpha power in the range higher than the PAF (e.g., 10 Hz) is spontaneously increased, ultimately reinforcing in a shift in the PAF.

In some embodiments, the patient the performs an action, for which a resulting positive feedback (e.g., reward due to decreased PPS and/or increased PAF) prompts the patient to perform the action again. As an exemplary analogy, the neurofeedback herein may be similar to a person learning how to walk, where positive reinforcements of an action prompt the person to continue doing such action in learning how to walk, where eventually such actions may occur naturally to the person. For example, in some cases, the patient may think of a pleasant memory, may perform deep breathing, may smile, or performs any type of action that previously resulted in a positive feedback. In some embodiments, the continual and/or periodic notification of a positive feedback (e.g., reward) for a given action may result in the patient subconsciously performing such action causing a shift in the pain sensitivity. In some embodiments, a plurality of actions, and/or permutations of such actions may result in positive feedback.

In some embodiments, an algorithm (e.g., executed by a processor) is configured to determine (e.g., select, calculate, etc.) the rewards and/or reward rate based on the PAF and/or PPS for the particular patient. The algorithm may include a statistical model, a predictive model, etc. In some embodiments, a machine learning (ML) model can be trained on subject data and the trained ML model can be used to determine the rewards and/or reward rate. For example, the subject data may include one or more characteristics of the subject including, e.g., health history, past PPS and/or PAF data, age, gender, etc. In some embodiments, the rewards and/or reward rate may be selected (e.g., via a user interface) by the subject and/or health care provider. In some embodiments, a patient may undergo a tuning exercise in which the reward, reward rate, and/or method of feedback (e.g., audio, visual, stimulation, etc.) are varied at set intervals. The variations in the reward and/or reward rate may be used as input features to an ML model. During this tuning evaluation, the change in PPS and/or PAF is used as an output to train an ML model using the input features. The input features can then be selected by the user, healthcare professional, or automatically to optimize the trained ML output of change in PPS.

A subject reaching a target change in the PPS and/or PAF can be used to determine whether the subject has modified pain sensitivity. In some embodiments, the target change in the PPS and/or PAF may be a percentage improvement in the subject's PPS and/or a percentage greater than the subject's PAF, respectively. The percentage may be at most 10%, at most 15%, at most 20%, at most 25%, at most 30%, or more. In another embodiment, the target change in the PPS and/or PAF may be an amount exceeding a threshold above the PAF and/or PPS. For example, the threshold may be predetermined based on the characteristics of the patient (e.g., type of chronic pain, underlying condition, past pain sensitivity data, etc.). In some embodiments, a signal from about 1-3 Hz, such as about 1-2 Hz, above the PAF correlates to an improvement in pain sensitivity correlating with a reward.

In process 206, the sensitivity can be optimized based on training including collecting EEG signals and providing feedback signals. As used herein, the term “training” may refer to a therapy, such as providing neurofeedback and/or providing entrainment (as described herein). The “optimization” of a subject's pain sensitivity may refer to the improvement of pain sensitivity and/or reduction in pain sensitivity. In some embodiments, the alpha power in the high frequency range (e.g., 10-12 Hz, also referred to as the fast range) is upregulated. This may be done with or without decreasing alpha power in the low frequency range (e.g., 8-10 Hz, also referred to as the slow range). This can result in a shift of the subject's PAF (e.g., from a lower frequency to a higher frequency) and/or an improvement in the subject's PPS.

FIG. 2B illustrates an example improvement in a subject's PPS (represented by line 214). Marker 216 indicates a subject's current PPS value and marker 218 indicates a subject's target PPS value. A subject's PPS value can move from her current PPS value 216 to a target PPS value 218 by shifting her brainwaves (e.g., spontaneously or with effort). As the PPS value moves, the subject is provided with a reward (e.g., a visual cue, an increased volume of an audio signal, etc.). The reward threshold 220 can be predetermined and/or may be based on the difference between the current PPS value 216 and the target PPS value 218. In some embodiments, the difference between the current PPS value 216 and the target PPS value 218 can be a percentage 222 or other points scale. In some embodiments, the reward is only provided based on positive changes in PPS (e.g., a decreasing PPS). In some embodiments, the reward includes points provided after a therapy session, wherein said points may be cumulative. The reward rate is the amount of feedback to provide to a subject based on how long the subject maintains her PPS above the reward threshold 220.

In some embodiments, the therapy provides a ramp-up reward methodology for a subject at the beginning (of a therapy session, or at the beginning of the entire therapy (e.g., at the first session)) and for a duration thereafter (ramp-up period). For example, in some embodiments, the subject may receive a reward (e.g., visual cue, auditory signal) when only achieving about 5%-15% of the difference between the current and target PPS values. As the subject continues to improve the PPS values, the rewards will be presented at higher intervals of PPS increase, such as about 15%-30%, 25%-50%4, 40%-60%, 50%-75%, 60%-85%, or 75%-99% of the difference between the current and target PPS values. In some embodiments, after the ramp-up period, the subject may only receive rewards after attaining the reward threshold (e.g., about 85%-100% of the difference between the current PPS value and target PPS value).

FIG. 2C provides example data of a subject's PAF before training (line 224), during training (line 226), and after training (line 228). The example PAF after training 228 was recorded 20 minutes after training. As depicted, the subject's PAF 226 increases while training (e.g., receiving feedback based on the subject's EEG signals). In this example training, the tone delivered was selected to be 0.5 Hz greater than the baseline PAF of 10.2 Hz. After training, the subject's PAF 228 appears to decrease to a frequency between the peaks 224 and 226.

In various embodiments, the signals associated with the feedback is provided to a computing system 208 (e.g., device, server, etc.), which may send the data to an interface accessible by a health care provider 210 and/or a patient 212. For example, the feedback may be in the form of an auditory, visual, haptic, electrical, magnetic, magnetic, and/or ultrasonic stimuli to the brain and/or peripheral nervous system of the subject. For instance, the feedback can be provided via a user interface of a mobile device (e.g., a smartphone, a tablet computer, etc.). In some cases, immediate (or near-immediate) feedback may help the patient improve his or her PPS and/or PAF more quickly.

In some embodiments, visual stimuli can be provided by a display screen of the mobile device. The visual stimuli can include changing a feature (e.g., color, shape, size, pattern, etc.) of an image on the display screen. For instance, when the PPS and/or PAF of a patient improves, the display screen can show a color change from red to yellow to green. Alternatively, the display screen can change the image itself from a less-pleasing image to a more-pleasing image. For example, in some embodiments, the image may become more brighter, correlating a with a more-pleasing image. In some embodiments, auditory stimuli can be provided by a speaker of the mobile device. The auditory stimuli may include a change in the loudness and/or pitch of the sound. For instance, the sound from the speaker of the mobile device can get increasingly louder and/or have increasingly higher pitch when the patient improves his or her PPS and/or PAF. In some embodiments, the auditory stimuli includes a frequency of a series of beeps that may progressively provided at a faster rate.

By seeing, hearing, and/or feeling a change in the feedback, the patient may be prompted to try to change the feedback further. In an example embodiment, the feedback may be part of a game that the patient can partake in. For instance, the display of a mobile device may illustrate racing objects (e.g., cars, runners, etc.). This type of feedback loop can tap into the natural competitiveness of a human to improve his or her pain sensitivity.

In some embodiments, to avoid the nocebo effect, the baseline pain sensitivity may be displayed (e.g., via the user interface) to the patient as a zero (0) and improvements in the patient's pain sensitivity are displayed as positive numbers (e.g., 1, 2, 3 . . . 10). In some instances, the patient is not told (e.g., via the interface or by the provider) that he or she is highly sensitive to pain but that the patient will benefit from the therapy. In some instances, a provider 210 may further use the sensitivity data to make treatment decisions including, e.g., prescribing medications, advising surgery, etc. for the patient.

In some embodiments, a patient's pain sensitivity data is analyzed periodically, intermittently, or continuously over a duration. For example, in treating endometriosis patients, the patient's PAF and/or PPS can be collected throughout the patient's menstrual cycle. The timing of the described feedback may accordingly be determined based on the proximity in time to menses. For example, in treating neuropathic pain, the patient's PAF and/or PPS can be collected on a daily or bi-weekly basis. Feedback may be applied in the morning or at time associated with the patient's greatest symptoms. For example, for patients having post-surgical or post-traumatic pain, therapy may be delivered prior to next scheduled pain dose, as needed (PRN), and/or scheduled daily if a given patient falls into a high pain sensitivity group, which may be at risk for the development of chronic pain. For example, for patients having chronic musculoskeletal pain (e.g., chronic lower back pain) suffering from osteoarthritis, the timing of the feedback may be related to the time at which their symptoms are worse (e.g., usually in the mornings).

In some embodiments, feedback is given asynchronously in which a patient receives rewards in the form of a visual cue (e.g., image of a beach or a pleasant animal) and/or an audio file when PPS is in a target state during real-time neurofeedback. Subsequent visual cues and/or audio files may be sent to the patient's mobile device at one or more time points (e.g., as described in the above examples) throughout the day to stimulate a shift in PAF or PPS.

### Entrainment

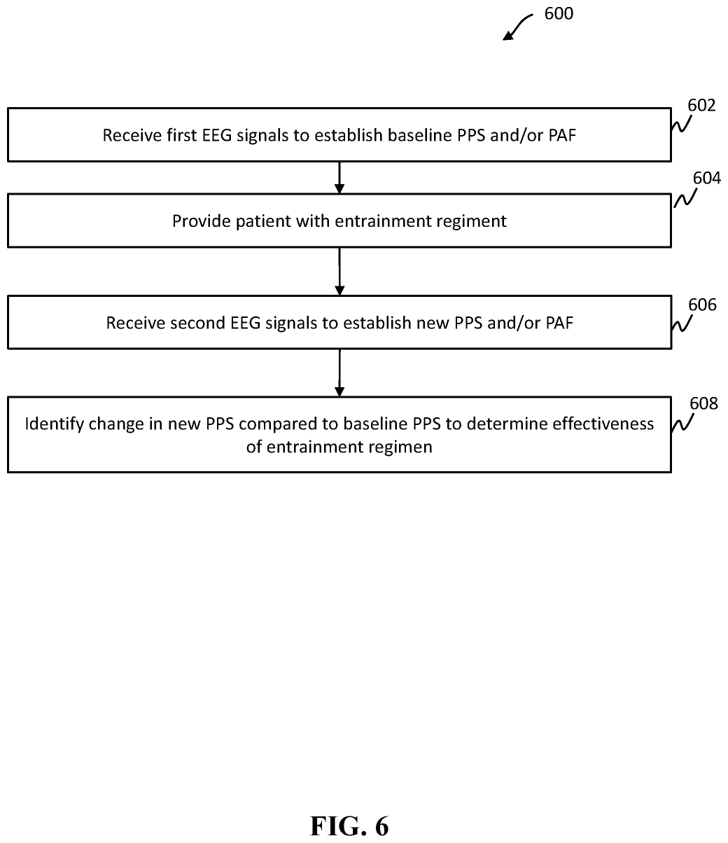

In some embodiments, in addition to or alternative to providing neurofeedback for modifying pain sensitivity, an entrainment method may be used to help modify pain sensitivity. FIG. 6 provides an exemplary embodiment of said entrainment method 600. In some embodiments, a patient obtains a baseline PPS and/or PAF 602 through collection of EEG data (e.g., first EEG signals). In some embodiments, the patient obtains the EEG data from a healthcare provider (e.g., physician, healthcare professional/administrator, other healthcare facility). In some embodiments, as described herein, the PPS is calculated based on the collected EEG data. In some embodiments, the PPS is calculated using an algorithm as described herein, which may take into account other trained data from other PPS and/or EEG data, and/or patient characteristics (e.g., age, health, gender, race, health condition, etc.). In some embodiments, based on the calculated baseline PPS, the patient is provided 604 with a prescribed entrainment regimen to help modify the pain sensitivity. In some embodiments, the prescribed entrainment regimen is tailored based on the subject, subject's characteristics, and/or the subject's PPS and/or EEG data. In some embodiments, the prescribed entrainment regimen is applicable to a large population, which may or may not be based on any tailored factors for the subject. This prescribed entrainment may be determined automatically from the users PPS with dosing (e.g. duration of entrainment of 10 minutes daily, or 20 minutes 3 times a week, or 20 minutes 3 days prior to menses) adjusted by monitoring PPS.

In some embodiments, the prescribed entrainment regimen comprises the patient receiving one or more audio and/or visual stimuli. In some embodiments, the audio stimuli and/or visual stimuli includes providing a sound that resonates with a PPS higher than the patient's baseline PPS. For example, in some embodiments, the audio stimuli comprises the patient listening to a volume of a tone, musical track, and/or other sound, which may be provided at a frequency from about 8 Hz to about 14 Hz, such as from about 9 Hz to about 13 Hz, or from about 10 Hz to about 12 Hz (so as to correlate to a PAF having a calculated PPS less than the baseline PPS). In some embodiments, the sound comprises a sub perceptible background tone to music with a frequency from about 8 Hz to about 14 Hz, such as from about 9 Hz to about 13 Hz, or from about 10 Hz to about 12 Hz. In some embodiments, the sound comprises a beat frequency, wherein two tones have a difference in frequency from about 8 Hz to about 14 Hz, such as from about 9 Hz to about 13 Hz, or from about 10 Hz to about 12 Hz. In some embodiments, the visual stimuli comprises a flicker and/or oscillation (e.g., on a smart device, such as a phone, TV, computing device, a light emitting device, etc.) at a frequency from about 8 Hz to about 14 Hz, such as from about 9 Hz to about 13 Hz, or from about 10 Hz to about 12 Hz. In some embodiments, the entrainment regimen comprises providing a vibrotactile stimulation at a frequency from about 8 Hz to about 14 Hz, such as from about 9 Hz to about 13 Hz, or from about 10 Hz to about 12 Hz.

In some embodiments, the entrainment method helps force the pain sensitivity to be modified based on a shift in the PAF.

In some embodiments, the entrainment regimen is provided a computing device, as described herein. For example, in some embodiments, the one or more video stimuli and/or the one or more audio stimuli are provided by a mobile device as described herein.

In some embodiments, after providing the prescribed entrainment regiment, the patient collects EEG data again 606 (e.g., second EEG signals) to calculate the respective PPS and/or determine the PAF, so as to identify a change in the calculated PPS as compared with the baseline PPS. In some embodiments, the first EEG signal in 602 is recorded at one visit, the entrainment regimen is performed at home, and then the second EEG signal (e.g. 606) to establish a new PPS and/or PAF (e.g. 608) is recorded at a follow up visit. In some embodiments, an effectiveness of the prescribed entrainment regimen is associated 608 based on the identified change in PPS. In some embodiments, the prescribed entrainment regiment is modified if i) an insufficient amount of change from the baseline PPS and/or ii) no change from the baseline PPS, was identified. In some embodiments, an increase in the measured PAF by at least about 0.04 Hz, 0.1 Hz, 0.25 Hz, or 0.5 Hz correlates with an effective entrainment regimen.

In some embodiments, the entrainment method is administered in combination with other treatments, such as a pharmacologic treatment. In some embodiments, the pharmacologic treatment comprises administering one or more centrally or peripherally acting neuromodulators to the patient during treatment, thereby modifying the pain sensitivity. In some embodiments, the entrainment method is provided to help wean off one or more pharmacologic agents. In some embodiments, the entrainment method is continued once the one or more pharmacologic agents is no longer received by the patient.

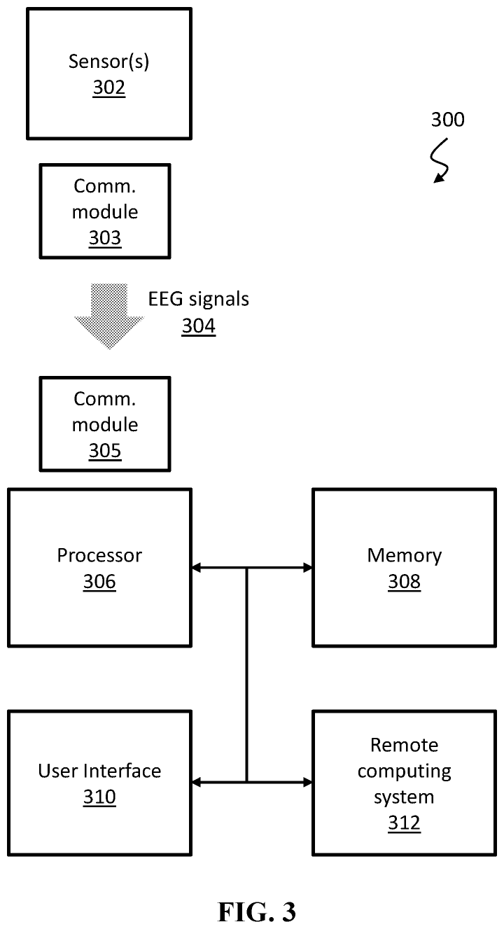

FIG. 3 is a diagram of an example system configured to modify pain sensitivity. The example system 300 includes one or more sensors 302 configured to detect EEG signals 304 from the human brain. In some embodiments, the sensors 302 may be superficial electrodes configured to be applied to the head of a human subject. In some embodiments, the sensors 302 may be part of a headband, a hat, or other item configured to be worn on the subject's head.

The system 300 can include one or more processors 306 configured to receive the EEG signals 304. For instance, the sensors 302 may be coupled to a communication module 303 configured to transmit the sensed EEG signals from to a communication module 305 coupled to the processor 306. As described further below, the processor 306 can provide feedback to the subject based on the detected EEG signals 304. The processor 306 may be communicatively coupled to a memory 308, which can be configured to store data (e.g., including the EEG signals 304). In some embodiments, the processor 306 and/or the memory 308 may be part of a computing device. The computing device may be a mobile phone, a smartphone, a tablet, a laptop computer, a notebook computer, a smartwatch, a set of smart glasses, a handheld computing device, a desktop computer, a server, a server system, etc.

The processor 306 and/or memory 308 may be communicatively coupled to the user interface 310, which can be configured to present information to the subject. The user interface 310 may be the interface of a mobile phone, a smartphone, a tablet, a laptop computer, a notebook computer, a smartwatch, a set of smart glasses, etc. The user interface 310 may be part of the same computing device as the processor 306 and/or memory 308. The processor 306, memory 308, and/or user interface 310 may be communicatively coupled to one or more remote computing systems 312 (e.g., a server system, a cloud, etc.). For example, data from a therapy session with the subject may be sent to and stored at a server. The data may then be available to physicians (e.g., remotely located physicians) for monitoring, determining treatment, etc. For instance, the data may be presented in a web application (e.g., accessible by the health care provider and/or subject). In another example, the data may be sent to a remote computing system 312 and become part of a patient's electronic medical record (EMR).

FIG. 4 is a flowchart of an example method 400 for modifying pain sensitivity. In step 402 of method 400, the processor 306 can receive a first set of EEG signals 304 from the sensors 302 (e.g., via communication modules 303 and/or 305). For example, the processor 306 may obtain 5 seconds or less, 8 seconds or less, 10 seconds or less, 15 seconds or less, or more of EEG signal duration from a given subject. In step 404, the processor 306 may determine, based on the first EEG signals 304, a first value for PPS and/or a second value for a PAF associated with the subject, as described above. This value for the PPS and/or value for the PAF may be used as a baseline for comparison to additionally determined values.

In step 406, the processor 306 can receive second EEG signals from the sensors. In some embodiments, the duration of received second EEG signals can be the same or different from the duration of received first EEG signals. In some embodiments, the second EEG signals can be continuously received (e.g., until the end of the training and/or therapy). In some embodiments, the processor 306 can determine a characteristic of the second EEG signal. For example, the characteristic can be the alpha power at a particular frequency. The characteristic of the second EEG signals can indicate reduced pain sensitivity when the processor 306 detects increased alpha power in the frequency range above (e.g., 0.5 Hz or less, 1 Hz or less, 1.5 Hz or less, 2 Hz or less, etc. greater than) the subject's PAF (e.g., baseline PAF based on the first EEG signals). In some embodiments, the processor 306 can determine a PPS value and/or PAF value based on the second EEG signals.

In step 408, the processor 306 can provide feedback (e.g., visual stimuli, auditory stimuli, etc.) to the subject when the characteristic of the second EEG signals indicates a reduced pain sensitivity. In some embodiments, as described, the characteristic can indicate reduced pain sensitivity when the alpha power in the frequency range above the subject's PAF has increased. In some embodiments, the processor 306 can provide feedback based on a comparison of the PPS and/or PAF associated with the first EEG signals to the PPS and/or PAF associated with the second EEG signals. When the comparison indicates reduced pain sensitivity, the processor 306 can provide the feedback signal.

In some embodiments, the processor 306 (or another processor) can determine whether the pain sensitivity of the subject is modified based on the characteristic, the first value, and/or the second value. For instance, the processor 306 can determine that the pain sensitivity is improved when the characteristic indicates reduced pain sensitivity (as described above). In another example, the processor 306 can determine that the pain sensitivity is improved based on a comparison of the PPS and/or PAF associated with the first EEG signals to the PPS and/or PAF associated with the second EEG signals. For example, when the value of the PPS associated with the second EEG signals is reduced (indicating a reduced pain sensitivity) in comparison to the value of the PPS associated with the first EEG signals, the processor 306 can determine that pain sensitivity is improved. For example, when the value of the PAF associated with the second EEG signals is increased (indicating a reduced pain sensitivity) in comparison to the value of the PAF associated with the first EEG signals, the processor 306 can determine that pain sensitivity is improved. In some embodiments, the processor 306 determines that the subject's pain sensitivity is modified when the subject maintains a reduced sensitivity state for a certain amount of time (e.g., up to 30 minutes, up to 1 hour, up to 3 hours, up to 6 hours, up to 12 hours, up to 1 day, up to 3 days, up to 1 week, etc.).

In some embodiments, the processor 308 can report to a user interface 310 whether the subject's pain sensitivity has improved. In some embodiments, further EEG signals (e.g., third EEG signals, fourth EEG signals, etc.) are received by the processor 306. The processor may determine, based on these further EEG signals, the PPS value and/or PAF value for the subject. These further values may be compared to respective values of, for example, the first EEG signals or the second EEG signals.

In some embodiments, the processor 306 can send additional feedback of the same type that was used during training at some later time. For example, if the feedback used in method 400 includes a pleasant sound and/or image, the processor 306 may send the same or similar pleasant sound and/or image to the mobile device of the subject at a later time (e.g., up to 30 minutes, up to 1 hour, up to 3 hours, up to 6 hours, up to 12 hours, up to 1 day, up to 3 days, up to 1 week, etc.). In this way, the subject may reenter the reduced pain sensitivity state based on the feedback prompt.

### Example 1: Effectiveness of Neurofeedback for Modifying Pain Sensitivity as Determined by Changes in Evoked Pain

Three subjects participated in 10 in-person neurofeedback therapy sessions performed in a crossover neurofeedback versus sham feasibility study. Sessions included quantitative sensory testing (QST), wherein warmth detection thresholds, heat pain thresholds, and heat pain tolerance are measured using a thermode (QST Lab Thermal Cutaneous Stimulator TCS II.1.b). A series of temperatures were applied to the skin on different dermatomes. EEG data (qEEG) was collected from 19 points of the 10-20 system using a Brainmaster Discovery neurological EEG. Data was recorded at 5 kHz with 0.016-250 Hz hardware filters and impedance at all recording electrodes will be maintained below 20 kW. Additional, qEEG measurements were taken with BrainBit 4 dry electrode headband.

The neurofeedback was performed using a Brainmaster EEG biofeedback equipment using sensor positions at 01, 02, T5, and T6. A baseline resting state EEG was recorded before and after neurofeedback sessions. The neurofeedback was targeting alpha power was increased in the frequency band 1-2 Hz above the peak alpha frequency determined by visual inspection of eyes closed resting state EEG.

The sham protocol was performed to provide a reward if the alpha power remained in the same state (e.g., no change).

Participants were blinded to sham or neurofeedback (they were not aware which feedback was increasing PAF or decreasing PPS).

QEEG was first performed with a 4 electrode dry system, which included one minute of eyes open and two minutes of eyes closed EEG data that will be recorded. Subsequent QEEG was performed with Brainmaster Discovery.

Quantitative Sensory Testing (QST) thermal threshold testing was performed in a testing room with simultaneous EEG recording. Testing was performed using a standard thermode. Each test was repeated three times with a break in between tests to familiarize participants with upcoming task and rating procedures.

A “levels” program determined the best temperature for phasic heat pain. This test involved 12 stimuli of various temperatures (ranging between 37-48° C.) with each trial lasting a total of 15 seconds. At the end of each stimulus, participants provided verbal pain ratings on a scale anchored from 0 (“no pain at all”) to 10 (“worst pain imaginable”).

Following the levels task, a single trial of phasic heat pain ratings task was performed to ensure the temperature is suitable for the participant. This test consisted of 40 seconds of stimulation using the temperature determined previously and 20 seconds of room temperature stimulation (˜32° C.). Data was collected using verbally reported ratings.

Prior to neurofeedback or sham, participants completed a single phasic heat pain protocol using the suitable temperature that was determined. Data was collected via a physical lab form completed by the experimenter.

Participants were randomized to either 5 sessions of sham or neurofeedback.

Participants were instructed that they will receive 10 sessions of neurofeedback that may affect their sensitivity to heat. Participants were asked at the end of the last session after QST “Did you believe you received the active condition or the sham condition in the last 5 sessions?”. During the study participants were told they are receiving neurofeedback during all sessions.

Neurofeedback or sham was performed at visits 1-10. QST were performed before neurofeedback/sham at Visits 1 and 6. QST were performed after neurofeedback/sham at Visits 5 and 10.

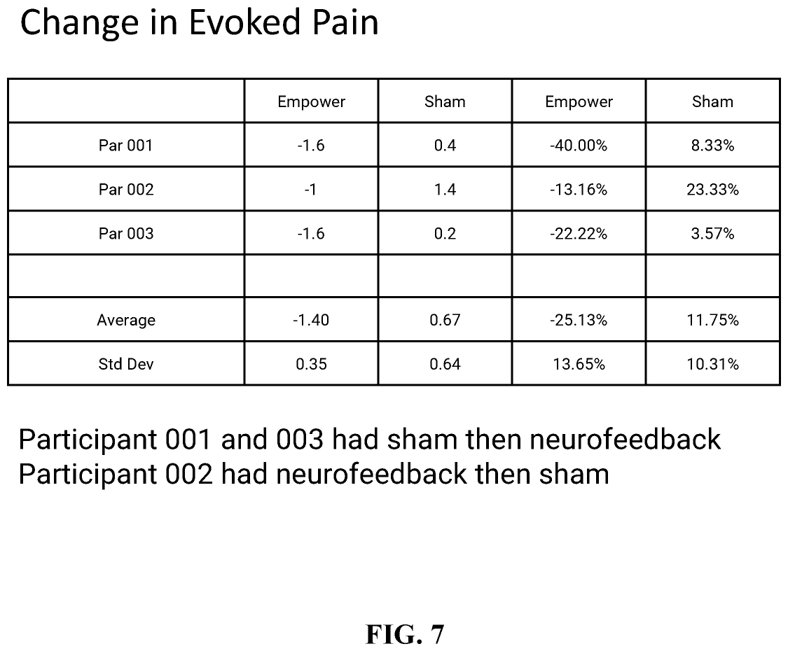

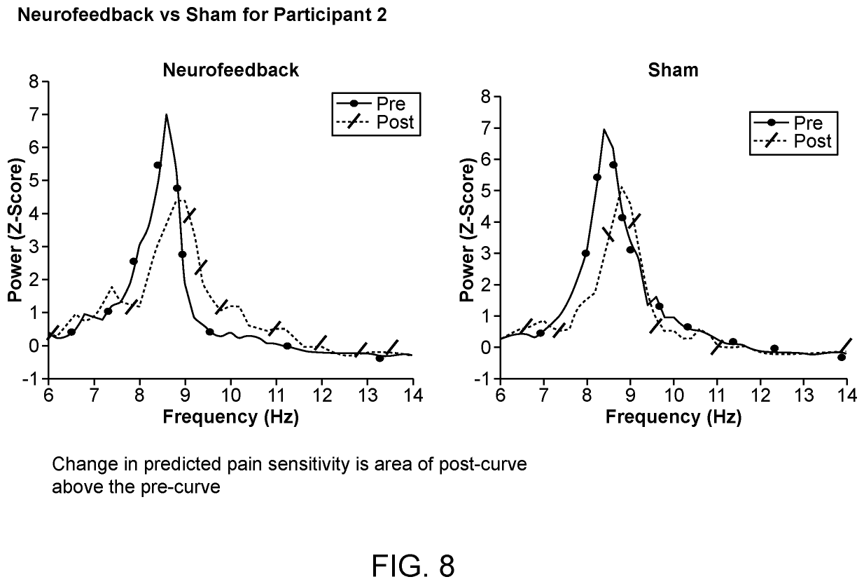

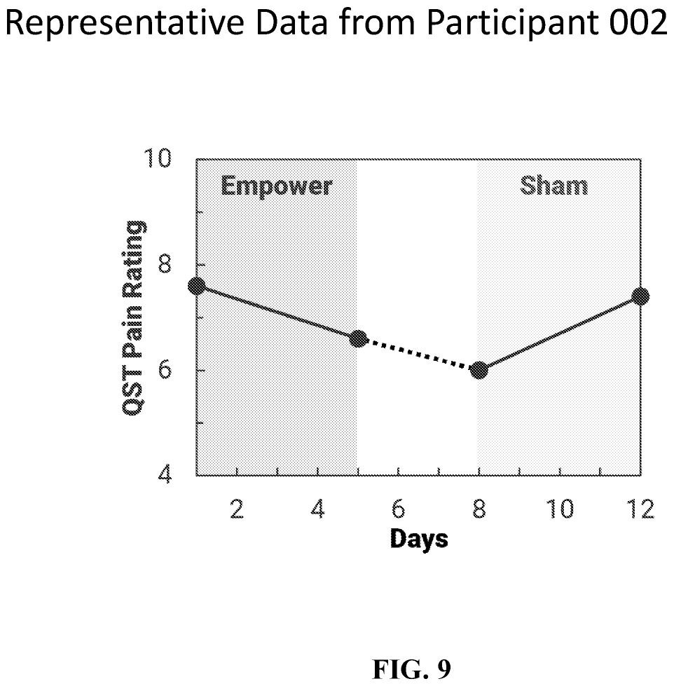

Results for 3 participants are provided in FIG. 7. Participants 001 and 003 had sham then neurofeedback, while Participant 002 had neurofeedback then sham. FIGS. 8-9 provide representative results for Participant 2, as described herein. For FIG. 8, a change in predicted pain sensitivity is identified as the area of post therapy curve over the pre therapy curve (e.g., see arrows on FIG. 8).

Participant 1: Participant 1 was with neuropathic pain, migraines, chronic depression, post partum depression, and PTSD. During the study there was a reduction in evoked pain of 40% during the neurofeedback session and increase in evoked pain of 8% during sham. Pain metrics were recorded via brief pain inventory. Pain severity changed from 8 to 4 where prior to the study the participant reported worst pain in the last 24 hours of 4/10 and 1/10 after the study. Pain interference in the week prior to the study was 3/10 for general activity, 7/10 for mood, walking ability 1/10 (did not interfere), normal work 4/10, relationships with people 8/10, sleep 8/10, and enjoyment of life 8/10. After the study was completed the participant reported no pain in the last 24 hours. Pain interference for the week during neurofeedback therapy was general activity 1/10, mood 1/10, walking ability 1/10, normal work 1/10, relationships with people 1/10, sleep 1/10, enjoyment of life 1/10. Composite pain interference score decreased from 5.6 to 1 consistent with improvements in quality of life.

Participant 2: Participant was with neuropathic pain, chronic lower back pain, headaches, knee pain, and anxiety. During the study there was a 13.2% reduction in evoked pain during neurofeedback and a 23.3% increase in evoked pain which happened during sham which followed the 5 neurofeedback sessions. At the end of the study pain severity changed from 29 to 28 and pain interference changed from 8.4 to 7.6, however this was after the participants sensitivity returned to baseline during sham. The week of neurofeedback therapy the participants' reported pain severity changed from 29 to 19 and pain interference improved from 8.4 to 4.4.

Participant 3: Participant was with depression, anxiety, chronic lower back pain, somatic symptoms and chronic fatigue disorder. During the study there was a 22.2% reduction in evoked pain with neurofeedback and a 3.6% increase in evoked pain with sham. Pain severity was 15 with worst pain in the last 24 hours of 8/10. Pain severity decreased to 6 with worst pain in the last 24 hours of 2/10 after 5 daily sessions of neurofeedback. Pain interference stayed the same from 1.7 to 1.7.

### Hardware and Software Implementations

In some examples, some or all of the processing described above can be carried out on a personal computing device, on one or more centralized computing devices, or via cloud-based processing by one or more servers. In some examples, some types of processing occur on one device and other types of processing occur on another device. In some examples, some or all of the data described above can be stored on a personal computing device, in data storage hosted on one or more centralized computing devices, or via cloud-based storage. In some examples, some data are stored in one location and other data are stored in another location. In some examples, quantum computing can be used. In some examples, functional programming languages can be used. In some examples, electrical memory, such as flash-based memory, can be used.

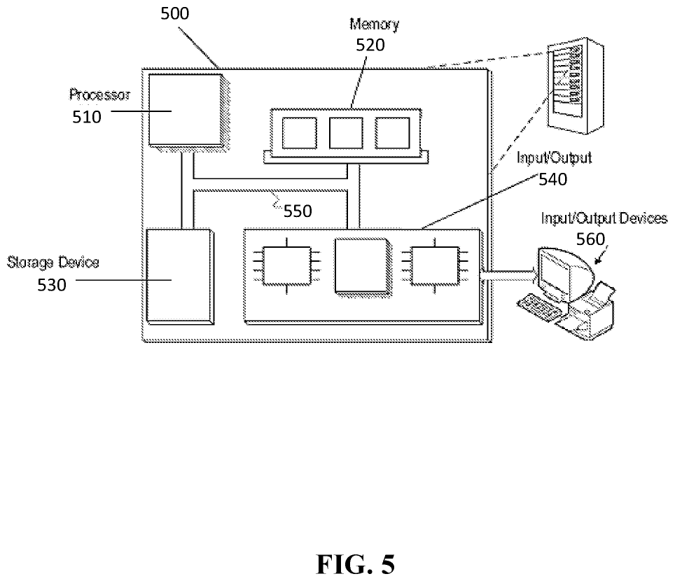

FIG. 5 is a block diagram of an example computer system 500 that may be used in implementing the technology described in this document. General-purpose computers, network appliances, mobile devices, or other electronic systems may also include at least portions of the system 500. The system 500 includes a processor 510, a memory 520, a storage device 530, and an input/output device 540. Each of the components 510, 520, 530, and 540 may be interconnected, for example, using a system bus 550. The processor 510 is capable of processing instructions for execution within the system 500. In some implementations, the processor 510 is a single-threaded processor. In some implementations, the processor 510 is a multi-threaded processor. The processor 510 is capable of processing instructions stored in the memory 520 or on the storage device 530.

The memory 520 stores information within the system 500. In some implementations, the memory 520 is a non-transitory computer-readable medium. In some implementations, the memory 520 is a volatile memory unit. In some implementations, the memory 520 is a non-volatile memory unit.

The storage device 530 is capable of providing mass storage for the system 500. In some implementations, the storage device 530 is a non-transitory computer-readable medium. In various different implementations, the storage device 530 may include, for example, a hard disk device, an optical disk device, a solid-date drive, a flash drive, or some other large capacity storage device. For example, the storage device may store long-term data (e.g., database data, file system data, etc.). The input/output device 540 provides input/output operations for the system 500. In some implementations, the input/output device 540 may include one or more of a network interface devices, e.g., an Ethernet card, a serial communication device, e.g., an RS-232 port, and/or a wireless interface device, e.g., an 802.11 card, a 3G wireless modem, or a 4G wireless modem. In some implementations, the input/output device may include driver devices configured to receive input data and send output data to other input/output devices, e.g., keyboard, printer and display devices 560. In some examples, mobile computing devices, mobile communication devices, and other devices may be used.

In some implementations, at least a portion of the approaches described above may be realized by instructions that upon execution cause one or more processing devices to carry out the processes and functions described above. Such instructions may include, for example, interpreted instructions such as script instructions, or executable code, or other instructions stored in a non-transitory computer readable medium. The storage device 530 may be implemented in a distributed way over a network, such as a server farm or a set of widely distributed servers, or may be implemented in a single computing device.

Although an example processing system has been described in FIG. 5, embodiments of the subject matter, functional operations and processes described in this specification can be implemented in other types of digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible nonvolatile program carrier for execution by, or to control the operation of, data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. The computer storage medium can be a machine-readable storage device, a machine-readable storage substrate, a random or serial access memory device, or a combination of one or more of them.

The term “system” may encompass all kinds of apparatus, devices, and machines for processing data, including by way of example a programmable processor, a computer, or multiple processors or computers. A processing system may include special purpose logic circuitry, e.g., an FPGA (field programmable gate array) or an ASIC (application specific integrated circuit). A processing system may include, in addition to hardware, code that creates an execution environment for the computer program in question, e.g., code that constitutes processor firmware, a protocol stack, a database management system, an operating system, or a combination of one or more of them.

A computer program (which may also be referred to or described as a program, software, a software application, a module, a software module, a script, or code) can be written in any form of programming language, including compiled or interpreted languages, or declarative or procedural languages, and it can be deployed in any form, including as a standalone program or as a module, component, subroutine, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Computers suitable for the execution of a computer program can include, by way of example, general or special purpose microprocessors or both, or any other kind of central processing unit. Generally, a central processing unit will receive instructions and data from a read-only memory or a random access memory or both. A computer generally includes a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few.

Computer readable media suitable for storing computer program instructions and data include all forms of nonvolatile memory, media and memory devices, including by way of example semiconductor memory devices, e.g., EPROM, EEPROM, and flash memory devices; magnetic disks, e.g., internal hard disks or removable disks; magneto optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's user device in response to requests received from the web browser.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

While this specification contains many specific implementation details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous. Other steps or stages may be provided, or steps or stages may be eliminated, from the described processes. Accordingly, other implementations are within the scope of the following claims.

### ADDITIONAL EMBODIMENTS

In one aspect, the disclosure features systems for modifying pain sensitivity in a subject. An example system can include a plurality of sensors configured to detect electroencephalography (EEG) signals in the subject, and a processor communicably coupled to the plurality of sensors. The plurality of sensors can receive first EEG signals from the sensors and determine, based on the first EEG signals, at least one of (i) a first value for a predicted pain sensitivity (PPS) associated with the subject or (ii) a second value for a peak alpha frequency (PAF) associated with the subject. The processor can be further configured to receive second EEG signals from the sensors, and provide feedback to the subject when a characteristic of the second EEG signals indicates a reduced pain sensitivity.

Various embodiments of the systems can include one or more of the following features.

The processor can be configured to determine that the pain sensitivity of the subject is modified based on at least one of the characteristic, the first value, or the second value. The PAF can be in a range of 8-12 Hz. The characteristic of the second EEG signals can include an alpha power value at a frequency, and the characteristic can indicate the reduced pain sensitivity when the alpha power value has increased at a frequency greater than the PAF. The processor can be configured to: receive third EEG signals from the sensors subsequent to providing feedback; determine, based on the third EEG signals, at least one of (i) a third value of the predicted pain sensitivity or (ii) a fourth value of the peak alpha frequency; and compare (i) the third value to the first value and/or (ii) the fourth value to the second value to determine whether the pain sensitivity in the subject is modified. Determining that the pain sensitivity is modified can be when the third value is a percentage greater the first value or the fourth value is a percentage greater than the second value. Determining that the pain sensitivity is modified can be when the third value is greater than a threshold above the first value or the fourth value is greater than a threshold above the second value.

The processor can be configured to: determine, based on the second EEG signals, at least one of (i) a third value of the predicted pain sensitivity or (ii) a fourth value of the peak alpha frequency; and compare (i) the third value to the first value and/or (ii) the fourth value to the second value; and provide the feedback based on the comparison. The feedback to the subject can include at least one of an auditory, visual, haptic, electrical, magnetic, or ultrasonic stimuli.

The system can include a mobile device configured to provide at least one of auditory stimuli or the visual stimuli. The system can include an apparatus configured to apply at least one of a magnetic stimuli or an ultrasonic stimuli to a brain or a peripheral nervous system of the subject.

The plurality of sensors can include electrodes configured to be positioned on a head of the subject. The predicted pain sensitivity can be based on Fourier transforms of the received first EEG signals or the second EEG signals in an alpha frequency range of 8-12 Hz. The first value of the predicted pain sensitivity can be based on the second value of the peak alpha frequency.

The predicted pain sensitivity can be a number on a predetermined scale. The system can include a first communication module coupled to the plurality of sensors and configured to transmit the first and second EEG signals; and a second communication module configured to receive the first and second EEG signals from the first communication module. The second communication module can be part of a mobile device. The processor can be part of the mobile device. The mobile device can include a user interface configured to present information based on the feedback signal. The processor can be part of a remote computing system. The remote computing system can include a storage module coupled to the processor and configured to store at least one of: (i) the first EEG signals, (ii) the second EEG signals, (iii) the first value for the predicted pain sensitivity, or (iv) the second value for the peak alpha frequency. The feedback can have a type, and the processor can be further configured to provide additional feedback of the type to the subject after determining that the pain sensitivity of the subject is modified.

The system can be configured to modify pain sensitivity associated with endometriosis in the subject. The system can be configured to modify pain sensitivity as an adjuvant therapy to endometriosis related central sensitization. The system can be configured to modify pain sensitivity associated with musculoskeletal pain in the subject. The system can be configured to modify pain sensitivity associated with chronic pain in the subject. The system can be configured to modify pain sensitivity associated with diabetic neuropathy in the subject. The system can be configured to modify pain sensitivity associated with shingles in the subject. The system can be configured to modify pain sensitivity associated with reflex sympathetic dystrophy syndrome in the subject. The system can be configured to modify pain sensitivity associated with cancer in the subject. The system can be configured to modify pain sensitivity associated with post-surgical pain in the subject. The system can be configured to modify pain sensitivity associated with a neurological disorder in the subject. The system can be configured to modify pain sensitivity associated with anxiety in the subject. The system can be configured to modify pain sensitivity associated with depression in the subject. The system can be configured to modify pain sensitivity associated with attention deficit hyperactivity disorder (ADHD) in the subject. The system is configured to prevent chronification of pain in the subject experiencing acute pain.

In another aspect, the disclosure features methods for modifying pain sensitivity in a subject. An example method can include receiving, by a processor from a plurality of sensors, first EEG signals and determining, by the processor based on the first EEG signals, at least one of (i) a first value for a predicted pain sensitivity (PPS) associated with the subject or (ii) a second value for a peak alpha frequency (PAF) associated with the subject. The example method can further include receiving, by a processor from a plurality of sensors, second EEG signals; and providing feedback to the subject when a characteristic of the second EEG signals indicates a reduced pain sensitivity.

Various embodiments of the methods can include one or more of the following features.

The method can include determining that the pain sensitivity of the subject is modified based on at least one of the characteristic, the first value, or the second value. The PAF is in a range of 8-12 Hz. The characteristic of the second EEG signals can include an alpha power value at a frequency, and the characteristic can indicate the reduced pain sensitivity when the alpha power value has increased at a frequency greater than the PAF. The method can include receiving third EEG signals from the sensors subsequent to providing feedback; determining, based on the third EEG signals, at least one of (i) a third value of the predicted pain sensitivity or (ii) a fourth value of the peak alpha frequency; and comparing (i) the third value to the first value and/or (ii) the fourth value to the second value to determine whether the pain sensitivity in the subject is modified. Determining that the pain sensitivity is modified can be when the third value is a percentage greater the first value or the fourth value is a percentage greater than the second value. Determining that the pain sensitivity is modified can be when the third value is greater than a threshold above the first value or the fourth value is greater than a threshold above the second value.

The method can include determining, based on the second EEG signals, at least one of (i) a third value of the predicted pain sensitivity or (ii) a fourth value of the peak alpha frequency; comparing (i) the third value to the first value and/or (ii) the fourth value to the second value to determine; and providing the feedback based on the comparison. The feedback to the subject can include at least one of an auditory, visual, haptic, electrical, magnetic, or ultrasonic stimuli. The feedback can be at least one of an auditory stimuli or a visual stimuli, and providing the feedback to the subject can be by a mobile device. The feedback can be at least one of a magnetic stimuli or an ultrasonic stimuli, and providing the feedback to the subject can be by an apparatus configured to apply the feedback. The plurality of sensors can include electrodes configured to be positioned on a head of the subject. The predicted pain sensitivity can be based on Fourier transforms of the received first EEG signals or the second EEG signals in an alpha frequency range of 8-12 Hz. The first value of the predicted pain sensitivity can be based on the second value of the peak alpha frequency.

The predicted pain sensitivity can be a number on a predetermined scale. The method can include transmitting, by a first communication module coupled to plurality of sensors, the first and second EEG signals; and receiving, by a second communication module, the first and second EEG signals from the first communication module. The second communication module can be part of a mobile device. The processor can be part of the mobile device. The mobile device can include a user interface configured to present information based on the feedback signal. The processor can be part of a remote computing system. The remote computing system can include a storage module coupled to the processor and configured to store at least one of: (i) the first EEG signals, (ii) the second EEG signals, (iii) the first value for the predicted pain sensitivity, or (iv) the second value for the peak alpha frequency. The feedback can have a type, and the method can further include providing additional feedback of the type to the subject after determining that the pain sensitivity of the subject is modified.

The method can be used to modify pain sensitivity associated with endometriosis in the subject. The method can be used to modify pain sensitivity as an adjuvant therapy to endometriosis related central sensitization. The method can be used to modify pain sensitivity associated with musculoskeletal pain in the subject. The method can be used to modify pain sensitivity associated with diabetic neuropathy in the subject. The method can be used to modify pain sensitivity associated with shingles in the subject. The method can be used to modify pain sensitivity associated with reflex sympathetic dystrophy syndrome in the subject. The method can be used to modify pain sensitivity associated with cancer in the subject. The method can be used to modify pain sensitivity associated with post-surgical pain in the subject. The method can be used to modify pain sensitivity associated with a neurological disorder in the subject. The method can be used to modify pain sensitivity associated with anxiety in the subject. The method can be used to modify pain sensitivity associated with depression in the subject. The method can be used to modify pain sensitivity associated with attention deficit hyperactivity disorder (ADHD) in the subject. The method is used to prevent chronification of pain in the subject experiencing acute pain.

### Terminology

The phraseology and terminology used herein is for the purpose of description and should not be regarded as limiting.

The term “approximately”, the phrase “approximately equal to”, and other similar phrases, as used in the specification and the claims (e.g., “X has a value of approximately Y” or “X is approximately equal to Y”), should be understood to mean that one value (X) is within a predetermined range of another value (Y). The predetermined range may be plus or minus 20%, 10%, 5%, 3%, 1%, 0.1%, or less than 0.1%, unless otherwise indicated.

The indefinite articles “a” and “an,” as used in the specification and in the claims, unless clearly indicated to the contrary, should be understood to mean “at least one.” The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The use of “including,” “comprising,” “having,” “containing,” “involving,” and variations thereof, is meant to encompass the items listed thereafter and additional items.

Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

What is claimed is:

1. A system for modifying pain sensitivity in a subject, the system comprising:
a plurality of sensors configured to detect electroencephalography (EEG) signals in the subject; and
a processor in operative communication with the plurality of sensors and configured to:
receive first EEG signals from the plurality of sensors;
determine, based on the first EEG signals, (i) a first predicted pain sensitivity (PPS) associated with the subject, and/or (ii) a first peak alpha frequency (PAF) associated with the subject;
receive second EEG signals from the sensors; and
provide feedback to the subject when a characteristic of the second EEG signals correlates with a second predicted pain sensitivity that is lower than the first predicted pain sensitivity.

2. The system of claim 1, wherein the processor is configured to determine that the pain sensitivity of the subject is modified based on at least one of the characteristic, the first PPS, and the first PAF.

3. The system of claim 1 or 2, wherein the first PAF is from about 8 Hz to about 12 Hz.

4. The system of any one of claims 1 to 3, wherein the characteristic of the second EEG signals comprises a second PAF associated with the subject, and wherein the second PPS is lower than the first PPS when the second PAF is greater than the first PAF.

5. The system of any one of claims 1 to 4, wherein the processor is configured to:
receive third EEG signals from the sensors subsequent to providing the feedback;
determine, based on the third EEG signals, (i) a third PPS, and/or (ii) a third PAF; and
compare (i) the third PPS to the first PPS, and/or (ii) the third PAF to the first PAF, to determine whether the pain sensitivity in the subject is modified.

6. The system of claim 5, wherein the modified pain sensitivity correlates with (i) the third PPS being lower than first PPS by a first minimum threshold, or (ii) the third PAF being greater than the first PAF by a second minimum threshold.

7. The system of claim 6, wherein the first minimum threshold and/or the second minimum threshold is from at least about 5% to at least about 40%.

8. The system of claim 6 or 7, wherein the second minimum threshold is from about at least about 0.01 Hz to about 1.0 Hz, such as at least about 0.02 Hz, 0.04 Hz, 0.05 Hz, 0.1 Hz, 0.25 Hz, 0.5 Hz, 0.75 Hz, or 1.0 Hz.

9. The system of any one of claims 1 to 8, wherein the processor determines the first PPS, the second PPS, and/or the third PPS based on one or more of age, gender, health history, past PPS and/or PAF data, etc.

10. The system of any one of claims 1 to 9, wherein the first PPS, the second PPS, and/or the third PPS is based on Fourier transforms of the received corresponding first EEG signals, the second EEG signals, and/or the third EEG signals, each in an alpha frequency range of about 8 Hz to about 12 Hz.

11. The system of any one of claims 1 to 10, wherein the first PPS is based on the first PAF.

12. The system of any one of claims 1 to 11, wherein the first PPS, the second PPS, and/or the third PPS is a number on a predetermined scale from 0 to 100.

13. The system of any one of claims 1 to 12, wherein the feedback to the subject comprises an auditory stimulus, a visual stimulus, a haptic stimulus, an electrical stimulus, a magnetic stimulus, an ultrasonic stimulus, or a combination thereof.

14. The system of claim 13, further comprising a computing device configured to provide at least one of the auditory stimulus and the visual stimulus.

15. The system of claim 14, wherein the computing device comprises a laptop, desktop, and/or a mobile device such as a smart phone, tablet, smartwatch, etc.

16. The system of claim 15, wherein the computing device comprises the processor.

17. The system of claim 15, wherein the computing device is in operative communication with the processor.

18. The system of any one of claims 13 to 17, wherein the auditory stimulus comprises a sound or tone having a prescribed loudness and/or pitch.

19. The system of claim 18, wherein the auditory stimulus comprises a change in a loudness and/or pitch of a sound or tone as compared with a previously provided sound or tone.

20. The system of claim any one of claims 13 to 18, wherein the visual stimulus comprises an image depicted on a display of the computing device.

21. The system of claim 20, wherein the visual stimulus comprises a change in a depicted image, wherein the change comprises a change in one or more of color, shape, size, and/or pattern of the depicted image.

22. The system of claim 20 or 21, wherein the visual stimulus comprises a change from a less pleasing image to a more pleasing image.

23. The system of any one of claims 13 to 22, further comprising an apparatus configured to apply at least one of a magnetic stimulus and/or an ultrasonic stimulus to a brain or a peripheral nervous system of the subject.

24. The system of any one of claims 1 to 23, wherein the feedback correlates only with a decreasing PPS.

25. The system of claim 24, wherein the feedback includes points based on the decreasing PPS that is optionally cumulative with each successive training session.

26. The system of any one of claims 1 to 25, wherein the plurality of sensors comprise electrodes configured to be positioned on a head of the subject.

27. The system of claim 26, wherein the plurality of sensors are provided with a headwear assembly, such as a headband, hat, or helmet.

28. The system of any one of claims 1 to 27, wherein the processor is part of a computing device.

29. The system of claim 28, wherein the computing device comprises a mobile device, a desktop, a laptop, and/or a remote computing system.

30. The system of claim 29, wherein the mobile device comprises a smartphone, a smart watch, a tablet, or any combination thereof.

31. The system of claim 30, wherein the computing device comprises and/or is operatively coupled to a storage module coupled to the processor and configured to store the first EEG signals, the second EEG signals, the third EEG signals, the first PPS, the second PPS, the third PPS, the first PAF, the second PAF, the third PAF, or a combination thereof.

32. The system of any one of claims 28 to 31, wherein the computing device comprises a user interface configured to provide the feedback and/or receive input from the subject.

33. The system of any one of claims 1 to 32, further comprising:
a first communication module coupled to the plurality of sensors and configured to transmit the first, second, and/or third EEG signals; and
a second communication module configured to receive the first, second, and/or third EEG signals from the first communication module.

34. The system of claim 33, wherein the second communication module is part of the computing device.

35. The system of any one of claims 1 to 34, wherein the feedback has a type, and wherein the processor is further configured to provide additional feedback of any type to the subject after determining that the pain sensitivity of the subject is modified.

36. The system of any one of claims 1 to 35, wherein the system is configured to modify pain sensitivity (i) associated with endometriosis in the subject, (ii) as an adjuvant therapy to endometriosis related central sensitization, (iii) associated with musculoskeletal pain in the subject, (iv) associated with diabetic neuropathy in the subject, (iv) associated with shingles in the subject, (v) associated with reflex sympathetic dystrophy syndrome in the subject, (vi) associated with cancer in the subject, (vii) associated with post-surgical pain in the subject, (viii) associated with a neurological disorder in the subject, (ix) associated with anxiety in the subject, (x) associated with depression in the subject, (xi) associated with attention deficit hyperactivity disorder (ADHD) in the subject, (xii) associated with post traumatic stress disorder (PTSD), (xiii) associated with nociplastic pain, (xiv) associated with chronic pelvis pain, (xv) associated with fibromyalgia, (xvi) associated with post traumatic pain, (xvii) associated with post surgical pain, or (xvii) any combination thereof.

37. The system of any one of claims 1 to 36, wherein the system is configured to prevent or reduce a chronification of pain in the subject experiencing acute pain.

38. The system of any one of claims 1 to 37, wherein the first EEG signals correspond to a lowest PPS score from a previous therapy session.

39. A method for modifying pain sensitivity in a subject, the method comprising:
a. receiving first EEG signals from a plurality of sensors coupled to the subject;
b. determining, based on the first EEG signals, (i) a first predicted pain sensitivity (PPS) associated with the subject, and/or (ii) a first peak alpha frequency (PAF) associated with the subject;
c. receiving second EEG signals from the plurality sensors; and
d. providing feedback to the subject when a characteristic of the second EEG signals correlates with a second predicted pain sensitivity that is lower than the first predicted pain sensitivity.

40. The method of claim 39, wherein the first PAF is from about 8 Hz to about 12 Hz.

41. The method of any one of claims 39 to 40, wherein the characteristic of the second EEG signals comprises a second PAF associated with the subject, and wherein the second PPS is lower than the first PPS when the second PAF is greater than the first PAF.

42. The method of any one of claims 39 to 41, further comprising:
a. receiving third EEG signals from the sensors subsequent to providing the feedback;
b. determining, based on the third EEG signals, (i) a third PPS, and/or (ii) a third PAF; and
c. comparing (i) the third PPS to the first PPS, and/or (ii) the third PAF to the first PAF, to determine whether the pain sensitivity in the subject is modified.

43. The method of claim 43, wherein the modified pain sensitivity correlates with (i) the third PPS being lower than first PPS by a first minimum threshold, or (ii) the third PAF being greater than the first PAF by a second minimum threshold.

44. The method of claim 43, wherein the first minimum threshold and/or the second minimum threshold is from at least about 5% to at least about 40%.

45. The method of claim 43 or 44, wherein the second minimum threshold is from about at least about 0.01 Hz to about 1.0 Hz, such as at least about 0.02 Hz, 0.04 Hz, 0.05 Hz, 0.1 Hz, 0.25 Hz, 0.5 Hz, 0.75 Hz, or 1.0 Hz.

46. The method of any one of claims 39 to 45, wherein the first PPS, the second PPS, and/or the third PPS is based on one or more of age, gender, health history, past PPS and/or PAF data, etc.

47. The method of any one of claims 39 to 46, wherein the first PPS, the second PPS, and/or the third PPS is based on Fourier transforms of the received corresponding first EEG signals, the second EEG signals, and/or the third EEG signals, each in an alpha frequency range of about 8 Hz to about 12 Hz.

48. The method of any one of claims 39 to 47, wherein the first PPS is based on the first PAF.

49. The method of any one of claims 39 to 48, wherein the first PPS, the second PPS, and/or the third PPS is a number on a predetermined scale from 0 to 100.

50. The method of any one of claims 39 to 49, wherein the feedback to the subject comprises an auditory stimulus, a visual stimulus, a haptic stimulus, an electrical stimulus, a magnetic stimulus, an ultrasonic stimulus, or a combination thereof.

51. The method of claim 50, wherein a computing device is configured to provide at least one of the auditory stimulus and the visual stimulus.

52. The method of claim 51, wherein the computing device comprises a laptop, desktop, and/or a mobile device such as a smart phone, tablet, smartwatch, etc.

53. The method of any one of claims 51 to 52, wherein the auditory stimulus comprises a sound or tone having a prescribed loudness and/or pitch.

54. The method of claim 53, wherein the auditory stimulus comprises a change in a loudness and/or pitch of a sound or tone as compared with a previously provided sound or tone.

55. The method of claim any one of claims 51 to 54, wherein the visual stimulus comprises an image depicted on a display of the computing device.

56. The method of claim 55, wherein the visual stimulus comprises a change in a depicted image, wherein the change comprises a change in one or more of color, shape, size, and/or pattern of the depicted image.

57. The method of claim 55 or 56, wherein the visual stimulus comprises a change from a less pleasing image to a more pleasing image.

58. The method of any one of claims 51 to 57, further comprising an apparatus configured to apply at least one of a magnetic stimulus and/or an ultrasonic stimulus to a brain or a peripheral nervous system of the subject.

59. The method of any one of claims 39 to 58, wherein the feedback correlates only with a decreasing PPS.

60. The method of claim 59, wherein the feedback includes points based on the decreasing PPS that is optionally cumulative with each successive training session.

61. The method of any one of claims 39 to 60, wherein the feedback has a type, and wherein the processor is further configured to provide additional feedback of any type to the subject after determining that the pain sensitivity of the subject is modified.

62. The method of any one of claims 39 to 61, wherein the method enables pain sensitivity modification (i) associated with endometriosis in the subject, (ii) as an adjuvant therapy to endometriosis related central sensitization, (iii) associated with musculoskeletal pain in the subject, (iv) associated with diabetic neuropathy in the subject, (iv) associated with shingles in the subject, (v) associated with reflex sympathetic dystrophy syndrome in the subject, (vi) associated with cancer in the subject, (vii) associated with post-surgical pain in the subject, (viii) associated with a neurological disorder in the subject, (ix) associated with anxiety in the subject, (x) associated with depression in the subject, (xi) associated with attention deficit hyperactivity disorder (ADHD) in the subject, (xii) associated with post traumatic stress disorder (PTSD), (xiii) associated with nociplastic pain, (xiv) associated with chronic pelvis pain, (xv) associated with fibromyalgia, (xvi) associated with post traumatic pain, (xvii) associated with post surgical pain, or (xvii) any combination thereof.

63. The method of any one of claims 39 to 62, wherein the method prevents or reduces chronification of pain in the subject experiencing acute pain.

64. The method of any one of claims 39 to 63, wherein the first EEG signals correspond to a lowest PPS score from a previous therapy session.

65. A non-transitory computer readable medium for modifying pain sensitivity in a subject, the non-transitory computer readable medium comprising instructions that, when executed by a processor, cause the processor to perform operations including:
a. receiving first EEG signals from a plurality of sensors coupled to the subject;
b. determining, based on the first EEG signals, (i) a first predicted pain sensitivity (PPS) associated with the subject, and/or (ii) a first peak alpha frequency (PAF) associated with the subject;
c. receiving second EEG signals from the plurality sensors; and
d. providing feedback to the subject when a characteristic of the second EEG signals correlates with a second predicted pain sensitivity that is lower than the first predicted pain sensitivity.

66. The non-transitory computer readable medium of claim 65, further comprising the processor in any one of claims 1 to 38.

67. The non-transitory computer readable medium of claim 65, wherein the non-transitory computer readable medium is part of a system of any one of claims 1 to 38.

68. A non-transitory computer readable medium for modifying pain sensitivity in a subject, the non-transitory computer readable medium comprising instructions that, when executed by a processor, cause the processor to perform operations including:
a. receiving first EEG signals from a plurality of sensors coupled to the subject;
b. determining, based on the first EEG signals, (i) a first predicted pain sensitivity (PPS) associated with the subject, and/or (ii) a first peak alpha frequency (PAF) associated with the subject;
c. correlating an entrainment regimen based on the first PPS and/or the first PAF; and
d. providing the entrainment regiment to the subject.

69. The non-transitory computer readable medium of claim 68, wherein the operations further include:
a. receiving second EEG signals from the plurality of sensors or a different plurality of sensors coupled to the subject;
b. determining, based on the second EEG signals, (i) a second PPS associated with the subject, and/or (ii) a second PAF associated with the subject; and
c. identifying an effectiveness of the entrainment regimen.

70. The non-transitory computer readable medium of claim 68 or 69, wherein the operations further includes modifying the entrainment regimen based on the identified effectiveness.

71. The non-transitory computer readable medium of any one of claims 68 to 70, wherein the identified effectiveness is based on i) the second PPS being lower than the first PPS by a first minimum threshold, and/or ii) the second PAF being greater than the first PAF by a second minimum threshold.

72. The non-transitory computer readable medium of claim 71, wherein the first minimum threshold and/or the second minimum threshold is from at least about 5% to at least about 40%.

73. The non-transitory computer readable medium of claim 71 or 72, wherein the second minimum threshold is from about at least about 0.01 Hz to about 1.0 Hz, such as at least about 0.02 Hz, 0.04 Hz, 0.05 Hz, 0.1 Hz, 0.25 Hz, 0.5 Hz, 0.75 Hz, or 1.0 Hz.

74. The non-transitory computer readable medium of any one of claims 68 to 73, wherein the entrainment regimen comprises one or more audio stimuli and/or one or more video stimuli.

75. The non-transitory computer readable medium of claim 74, wherein the one or more audio stimuli comprises a volume of a tone, musical track, and/or other sound at a prescribed frequency based on the first PAF and/or the first PPS.

76. The non-transitory computer readable medium of claim 74 or 75, wherein the one or more audio stimuli comprises sub perceptible background tone at the prescribed frequency.

77. The non-transitory computer readable medium of any one of claims 74 to 76, wherein the one or more audio stimuli comprises a beat frequency wherein two tones have a difference in frequency of the prescribed frequency.

78. The non-transitory computer readable medium of any one of claims 74 to 77, wherein the one or more video stimuli comprises a flicker and/or oscillation provided on a display device, wherein the flicker and/or oscillation is provided at the prescribed frequency.

79. The non-transitory computer readable medium of any one of claims 74 to 78, wherein the one or more video stimuli comprises a flicker and/or oscillation provided on a display device, wherein the flicker and/or oscillation is provided at the prescribed frequency.

80. The non-transitory computer readable medium of any one of claims 68 to 79, wherein the entrainment regimen comprises providing a vibrotactile stimulation at the prescribed frequency.

81. The non-transitory computer readable medium of any one of claims 68 to 80, wherein the prescribed frequency is based on a minimum PAF that correlates with a PPS greater than the first PPS.

82. The non-transitory computer readable medium of any one of claims 68 to 81, wherein the prescribed frequency is from about 10 Hz to about 12 Hz.

83. The non-transitory computer readable medium of any one of claims 68 to 82, wherein the processor is part of a computing device.

84. The non-transitory computer readable medium of claim 83, wherein the computing device comprises a mobile device, a desktop, a laptop, and/or a remote computing system.

85. The non-transitory computer readable medium of claim 84, wherein the mobile device comprises a smartphone, a smart watch, a tablet, or any combination thereof.

86. The non-transitory computer readable medium of any one of claims 83 to 85, wherein the computing device comprises and/or is operatively coupled to a storage module coupled to the processor and configured to store the first EEG signals, the second EEG signals, the first PPS, the second PPS, the first PAF, the second PAF, or a combination thereof.

87. The non-transitory computer readable medium of any one of claims 83 to 86, wherein the computing device comprises a user interface configured to provide the feedback and/or receive input from the subject.

88. The non-transitory computer readable medium of any one of claims 83 to 87, wherein the entrainment regimen is provided by the computing device.

89. The non-transitory computer readable medium of any one of claims 68 to 88, wherein the processor determines the first PPS and/or the second PPS based on one or more of age, gender, health history, past PPS and/or PAF data, etc.

90. The non-transitory computer readable medium of any one of claims 68 to 89, wherein the first PPS and/or the second PPS is based on Fourier transforms of the received corresponding first EEG signals and/or the second EEG signals, each in an alpha frequency range of about 8 Hz to about 12 Hz.

91. The non-transitory computer readable medium of any one of claims 68 to 90, wherein the first PPS is based on the first PAF.

92. The non-transitory computer readable medium of any one of claims 68 to 91, wherein the first PPS and/or the second PPS is a number on a predetermined scale from 0 to 100.

93. The non-transitory computer readable medium of any one of claims 68 to 92, wherein the plurality of sensors comprise electrodes configured to be positioned on a head of the subject.

94. The non-transitory computer readable medium of claim 93, wherein the plurality of sensors are provided with a headwear assembly, such as a headband, hat, or helmet.

95. The non-transitory computer readable medium of any one of claims 83 to 94, wherein the computing device comprises and/or is operatively coupled to a storage module coupled to the processor and configured to store the first EEG signals, the second EEG signals, the third EEG signals, the first PPS, the second PPS, the third PPS, the first PAF, the second PAF, the third PAF, or a combination thereof.

96. The non-transitory computer readable medium of any one of claims 83 to 95, wherein the computing device comprises a user interface configured to receive input from the subject.

97. The non-transitory computer readable medium of any one of claims 68 to 96, further comprising:
a first communication module coupled to the plurality of sensors and configured to transmit the first and/or second EEG signals; and
a second communication module configured to receive the first and/or second EEG signals from the first communication module.

98. The non-transitory computer readable medium of claim 97, wherein the second communication module is part of the computing device.

99. The non-transitory computer readable medium of any one of claims 68 to 98, wherein the non-transitory computer readable medium is configured to modify pain sensitivity (i) associated with endometriosis in the subject, (ii) as an adjuvant therapy to endometriosis related central sensitization, (iii) associated with musculoskeletal pain in the subject, (iv) associated with diabetic neuropathy in the subject, (iv) associated with shingles in the subject, (v) associated with reflex sympathetic dystrophy syndrome in the subject, (vi) associated with cancer in the subject, (vii) associated with post-surgical pain in the subject, (viii) associated with a neurological disorder in the subject, (ix) associated with anxiety in the subject, (x) associated with depression in the subject, (xi) associated with attention deficit hyperactivity disorder (ADHD) in the subject, (xii) associated with post traumatic stress disorder (PTSD), (xiii) associated with nociplastic pain, (xiv) associated with chronic pelvis pain, (xv) associated with fibromyalgia, (xvi) associated with post traumatic pain, (xvii) associated with post surgical pain, or (xvii) any combination thereof.

100. The non-transitory computer readable medium of any one of claims 68 to 99, wherein the non-transitory computer readable medium is configured to prevent or reduce a chronification of pain in the subject experiencing acute pain.

101. The non-transitory computer readable medium of any one of claims 68 to 100, wherein the first EEG signals correspond to a lowest PPS score from a previous therapy session.

102. A method for modifying pain sensitivity in a subject, the method comprising:
a. receiving first EEG signals from a plurality of sensors coupled to the subject;
b. determining, based on the first EEG signals, (i) a first predicted pain sensitivity (PPS) associated with the subject, and/or (ii) a first peak alpha frequency (PAF) associated with the subject; and
c. correlating an entrainment regimen based on the first PPS and/or the first PAF; and
d. providing the entrainment regiment to the subject.

103. The method of claim 102, further comprising:
a. receiving second EEG signals from the plurality of sensors or a different plurality of sensors coupled to the subject;
b. determining, based on the second EEG signals, (i) a second PPS associated with the subject, and/or (ii) a second PAF associated with the subject; and
c. identifying an effectiveness of the entrainment regimen.

104. The method of claim 102 or 103, further comprising modifying the entrainment regimen based on the identified effectiveness.

105. The method of any one of claims 102 to 104, wherein the identified effectiveness is based on i) the second PPS being lower than the first PPS by a first minimum threshold, and/or ii) the second PAF being greater than the first PAF by a second minimum threshold.

106. The method of claim 105, wherein the first minimum threshold and/or the second minimum threshold is from at least about 5% to at least about 40%.

107. The method of claim 105 or 106, wherein the second minimum threshold is from about at least about 0.01 Hz to about 1.0 Hz, such as at least about 0.02 Hz, 0.04 Hz, 0.05 Hz, 0.1 Hz, 0.25 Hz, 0.5 Hz, 0.75 Hz, or 1.0 Hz.

108. The method of any one of claims 102 to 107, wherein the entrainment regimen comprises one or more audio stimuli and/or one or more video stimuli.

109. The method of claim 108, wherein the one or more audio stimuli comprises a volume of a tone, musical track, and/or other sound at a prescribed frequency based on the first PAF and/or the first PPS.

110. The method of claim 108 or 109, wherein the one or more audio stimuli comprises sub perceptible background tone at the prescribed frequency.

111. The method of any one of claims 108 to 110, wherein the one or more audio stimuli comprises a beat frequency wherein two tones have a difference in frequency of the prescribed frequency.

112. The method of any one of claims 108 to 111, wherein the one or more video stimuli comprises a flicker and/or oscillation provided on a display device, wherein the flicker and/or oscillation is provided at the prescribed frequency.

113. The method of any one of claims 108 to 112, wherein the one or more video stimuli comprises a flicker and/or oscillation provided on a display device, wherein the flicker and/or oscillation is provided at the prescribed frequency.

114. The method of any one of claims 102 to 113, wherein the entrainment regimen comprises providing a vibrotactile stimulation at the prescribed frequency.

115. The method of any one of claims 102 to 114, wherein the prescribed frequency is based on a minimum PAF that correlates with a PPS greater than the first PPS.

116. The method of any one of claims 102 to 115, wherein the prescribed frequency is from about 10 Hz to about 12 Hz.

117. The method of any one of claims 102 to 116, wherein the first PPS and/or the second PPS is based on one or more of age, gender, health history, past PPS and/or PAF data, etc.

118. The method of any one of claims 102 to 117, wherein the first PPS and/or the second PPS is based on Fourier transforms of the received corresponding first EEG signals and/or the second EEG signals, each in an alpha frequency range of about 8 Hz to about 12 Hz.

119. The method of any one of claims 102 to 118, wherein the first PPS is based on the first PAF.

120. The method of any one of claims 102 to 119, wherein the first PPS and/or the second PPS is a number on a predetermined scale from 0 to 100.

121. The method of any one of claims 102 to 120, wherein the method enables pain sensitivity modification (i) associated with endometriosis in the subject, (ii) as an adjuvant therapy to endometriosis related central sensitization, (iii) associated with musculoskeletal pain in the subject, (iv) associated with diabetic neuropathy in the subject, (iv) associated with shingles in the subject, (v) associated with reflex sympathetic dystrophy syndrome in the subject, (vi) associated with cancer in the subject, (vii) associated with post-surgical pain in the subject, (viii) associated with a neurological disorder in the subject, (ix) associated with anxiety in the subject, (x) associated with depression in the subject, (xi) associated with attention deficit hyperactivity disorder (ADHD) in the subject, (xii) associated with post traumatic stress disorder (PTSD), (xiii) associated with nociplastic pain, (xiv) associated with chronic pelvis pain, (xv) associated with fibromyalgia, (xvi) associated with post traumatic pain, (xvii) associated with post surgical pain, or (xvii) any combination thereof.

122. The method of any one of claims 102 to 121, wherein the method is configured to prevent or reduce a chronification of pain in the subject experiencing acute pain.

123. The method of any one of claims 102 to 122, wherein the first EEG signals correspond to a lowest PPS score from a previous therapy session.

124. The method of any one of claims 102 to 124, wherein the entrainment regiment is provided using a computing device.

125. The method of claim 124, wherein the computing device comprises a mobile device, a desktop, a laptop, and/or a remote computing system.

126. The non-transitory computer readable medium of claim 125, wherein the mobile device comprises a smartphone, a smart watch, a tablet, or any combination thereof.
